SEQUENCE LISTING (30 sequences)

SEQ ID NO: 1 - DNA, 1505 bp, Artificial Sequence
  Description of Artificial Sequence: Synthetic polynucleotide
ggaaataagagagaaaagaagagtaagaagaaatataagagccaccatgaagtgaggctctcctcccgccccgcccctcccacgcctcaccagccccccgcgcgcccaccctccggcgggtgacagctccgggatcagcaacccttcctgctgctgctactgctgctgctgctgccgccgccgccgccgccgctgcccttgggtccccccgagtctccgggactgccctctcgactgtcagtggggcagcctctccgactctgcacccgcctcgacctccccacccgctcccacacccctgtgccctcatgtggagcctaagagaacagaacaggccgtgaagccagcagagaaaggaagcggagctactaacttcagcctgctgaagcaggctggagacgtggaggagaaccctggaccttactcgagcagacgcgtagatctgggctccggcgagggcaggggaagtcttctaacatgcggggacgtggaggaaaatcccggcccaggcagcgccttcgagcgcgtcgtccgccgcgtcgtccaggagctcgaccacggcggcgagttcatccccgtcaccagcctccagagcagcaccggcttccagccctactgcctcgtcgtccgcaagcccagcagcagctggttctggaagccccgctacaagtgcgtgaacctgagcatcaaggacatcctcgagccggacgccgccgagcccgacgtccagcgcggccgcagcttccacttctacgacgccatggacggccagatccagggcagcgtcgagctggccgcccccggccaggccaagatcgccggcggcgccgccgtcagcgacagcagcagcaccagcatgaacgtctacagcctcagcgtcgaccccaacacctggcagaccctcctccacgagcgccacctccgccagcccgagcacaaggtcctccagcagctccgcagccggggcgacaacgtctacgtcgtcaccgaggtcctccagacccagaaggaggtcgaggtcacccggacccacaagcgcgagggcagcggccgcttcagcctccccggcgccacctgcctccagggcgagggccagggccacctcagccagaagaagaccgtcaccatccccagcggcagcaccctcgccttccgcgtcgcccagctcgtcatcgacagcgacctcgacgtcctcctcttccccgacaagaagcagcgcaccttccagccccccgccaccggccacaagcgcagcaccagcgagggcgcctggccccagctccccagcggcctcagcatgatgcgctgcctccacaacttcctcaccgactgataattaaaccggtttaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgcgctagcgctg

SEQ ID NO: 2 - DNA, 1663 bp, Artificial Sequence
  Description of Artificial Sequence: Synthetic polynucleotide
ggtcaagcttttggaccctcgtacagaagctaatacgactcactatagggaaataagagagaaaagaagagtaagaagaaatataagagccaccatggaaaatttgaagcatattatcacccttggccaggtcatccacaaacggtgtgaagagatgaaatactgcaagaaacagtgccggcgcctgggccaccgcgtcctcggcctgatcaagcctctggagatgctccaggaccaaggaaagaggagcgtgccctctgagaagttaaccacagccatgaaccgcttcaaggctgccctggaggaggctaatggggagatagaaaagttcagcaatagatccaatatctgcaggtttctaacagcaagccaggacaaaatactcttcaaggacgtgaacaggaagctgagtgatgtctggaaggagctctcgctgttacttcaggttgagcaacgcatgcctgtttcacccataagccaaggagcgtcctgggcacaggaagatcagcaggatgcagacgaagacaggcgagctttccagatgctaagaagagataatgaaaaaatagaagcttcactgagacgattagaaatcaacatgaaagaaatcaaggaaactttgaggcagtatttaccaccaaaatgcatgcaggagatcccgcaagagcaaatcaaggagatcaagaaggagcagctttcaggatccccgtggattctgctaagggaaaatgaagtcagcacactttataaaggagaataccacagagctccagtggccataatggtattcaaaaaactccaggctggcagcattgcaatagtgaggcagactttcaataaggagatcaaaaccatgaagaaattcgaatctcccaacatcctgcgtatatttgggatttgcattgatgaaacagtgactccgcctcaattctccattgtcatggagtactgtgaactcgggaccctgagggagctgttggatagggaaaaagacctcacacttggcaagcgcatggtcctagtcctgggggcagcccgaggcctataccggctacaccattcagaagcacctgaactccacggaaaaatcagaagctcaaacttcctggtaactcaaggctaccaagtgaagcttgcaggatttgagttgaggaaaacagccacttccatgagtttgggaactacgagagaaaagacagacagagtcaaatctacagcatatctctcacctcaggaactggaagatgtattttatcaatatgatgtaaagtctgaaatatacagctttggaatcgtcctctgggaaatcgccactggagatatcccgtttcaaggctgtaattctgagaagatccgcaagctggtggctgtgaagcggcagcaggagccactgggtgaagactgcccttcagagctgcgggagatcattgatgagtgccgggcccatgatccctctgtgcggccctctgtggatgaaatcttaaagaaactctccaccttttctaagtgattaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgc

SEQ ID NO: 3 - DNA, 1027 bp, Artificial Sequence
  Description of Artificial Sequence: Synthetic polynucleotide
ggaaataagagagaaaagaagagtaagaagaaatataagagccaccatggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctgcagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagattgattaattaagctgccttctgcggggcttgccttctggccatgcccttcttctctcccttgcacctgtacctcttggtctttgaataaagcctgagtaggaagaagcctgcatgcctggttctctgcgtctgcgaattcgatatccagcggccgc

SEQ ID NO: 4 - DNA, 3270 bp, Artificial Sequence
  Description of Artificial Sequence: Synthetic polynucleotide
gaaataagagagaaaagaagagtaagaagaaatataagagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgggctccggcgagggcaggggaagtcttctaacatgcggggacgtggaggaaaatcccggcccaatggataaattgggacagatcatcaagttaggccagctcatctatgaacagtgtgaaaagatgaaatactgccggaaacaatgccagcgtctaggaaaccgtgtgcacggcctgctacagcctctccagagactccaggcccaaggaaagaagaacctgcccgatgacattactgctgccctgggccgttttgatgaagtcctgaaggaggctaaccagcagatagaaaagttcagcaagaagtcccatatttggaagtttgtgagtgtgggcaatgataagatcctcttccatgaagtgaatgagaagctgagagacgtctgggaggagctgttgctgctgcttcaggtttatcattggaataccgtttcagatgtcagccagccagcatcctggcagcaggaagatcgacaggatgcagaggaagacggaaatgaaaatatgaaagttatcctgatgcagttgcaaattagcgtggaagaaatcaacaaaaccctgaagcaatgctcactaaaacccacacaggagatcccacaagatctccaaatcaaggagattccaaaggaacatcttggacctccgtggaccaaactgaagacaagtaaaatgagcaccatttatagaggagagtatcacagatctccagttaccatcaaagtattcaacaacccccaggccgaaagtgttggaatagtgaggttcactttcaatgacgagatcaaaaccatgaagaaattcgattctcccaacatcttgcgtatatttgggatttgcattgatcaaacagtgaagccccctgagttctccattgtcatggagtactgtgaacttggaaccctgagggaactgctggatagagaaaaagacctcacaatgagtgtgcgcagcctcctagtcctgagggcagccagaggcttatacaggctacaccattcggaaacactccacagaaacatcagcagctccagtttcctcgtagccggaggctaccaagtaaagcttgcaggatttgagttaagcaaaacacagaatgatatcgaccggacagcaaagagcactaaagcagagagatccagttcaacgatatatgtctcccctgagagactgaaaaatccattttgcctttatgacataaaagctgaaatatatagctttggaattgtactctgggaaattgccactggaaagatcccatttgaaggctgtgattctaagaagatccgggagctggtggctgaggacaagaagcaggaaccagtgggtcaggattgccctgagttgttgcgggaaatcattaatgagtgtcgtgcccatgagccctcccaacggccctctgtggacggtaggagtctttctggcagagaacgaatcttggagagactgtctgcggttgaagaatccacggacaagaaggtgtaagctgccttctgcggggcttgccttctggccatgcccttcttctctcccttgcacctgtacctcttggtctttgaataaagcctgagtaggaag

SEQ ID NO: 5 - DNA, 2069 bp, Artificial Sequence
  Description of Artificial Sequence: Synthetic polynucleotide
aggtcttctggtccccacagactcagagagaacccgccaccatgagcagcgtgaagctgtggcctaccggcgccagcgccgtgcccctggtgagccgggaggagttaaagaagctggagttcgtgggcaagggcggcttcggcgtggtgttccgggcccaccaccggacttggaaccacgatgtggccgtgaagatcgtgaatagcaagaagatcagctgggaggtgaaggccatggtgaacctgcggaacgagaacgtgctgctgctgctgggcgtgaccgaggacctgcagtgggacttcgtgagcggccaggccctggtgacccggttcatggagaatggcagcctggccggcctgctgcagcccgagtgccctcggccctggcccctcctgtgccggctgctgcaggaggtggtgctgggcatgtgctacctgcacagcctgaacccacccctgctgcacagggacctgaagcccagcaacatcctgctggaccctgagctgcacgccaagctggccgatttcggcctgagcaccttccagggcggcagccagagcggcagcggcagcggaagcggctcacgggacagcggcggcaccctggcctacctggaccccgagctgctgttcgacgtgaacctgaaggccagcaaagccagcgacgtgtacagcttcggcatcctggtgtgggccgtgctggcaggccgggaggctgagctggtggacaagaccagcctgatccgggagaccgtgtgcgaccggcagagccggccccccctgaccgagctgccccccggcagccccgagacccccggcctggagaagctcaaggagctgatgatccactgctggggctcacagtccgagaatcgtccaagcttccaggactgcgagcccaagaccaacgaggtgtacaacctggtgaaggacaaggtggacgccgccgtgagcgaggtgaagcactacctgagccagcaccggagcagcggccggaacctgagcgcccgggagcccagccagcggggcaccgagatggactgcccccgggagaccatggtgagcaagatgctggaccggctgcacctggaggagcccagcggacctgtgcccggcaagtgccccgagcggcaggcccaggacaccagcgtgggccccgccacccccgcccggaccagcagcgaccccgtggccggcacccctcagatcccccacaccctgcccttccggggcaccacccccggccccgtgttcaccgagaccccaggcccccacccccagaggaatcagggcgacggccggcacggcaccccatggtacccttggaccccccccaaccccatgaccggcccccccgcactggtgtttaataactgtagcgaggtgcagatcggcaactacaacagcctggtcgcccccccccggaccaccgccagcagcagcgccaagtacgaccaggcccagttcggccggggccggggctggcagcccttccacaagggcaccaagtacgagatcgaacggatgctgaaggagctgaaggacctgctggaagagctgaagcggatgctggaggagcttaaacggagcctgaaggaactgaagaagaaccccagcgaggacgccctggtggagaataaccggctgatcgtgaaggtgctggaggtgatcgtggaggtgctgcgggccatcatcgagctggccgagctgatcatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 6 - DNA, 2165 bp, Artificial Sequence
  Description of Artificial Sequence: Synthetic polynucleotide
aggtcttctggtccccacagactcagagagaacccgccaccatgagctgcgtgaagctgtggcccagcggcgcccccgccccactggtgagcatcgaggagctggagaaccaggagctggtgggcaagggcggcttcggcaccgtgttccgggcccagcaccggaagtggggctacgacgtggccgtgaagatcgtgaacagcaaggcaatcagccgggaggtgaaggccatggccagcctggacaacgagttcgtgctgcggctggagggcgtgatcgagaaggtgaactgggaccaggacccaaagcctgccctggtgaccaagttcatggagaacggcagcctgagcggactgctgcagagccagtgcccccggccctggccactgctgtgccggctgctgaaggaggtggtgctgggcatgttctacctgcacgatcagaaccccgtgctgctgcaccgtgacctgaagcccagcaacgtgctgctggaccccgagctgcacgtgaagctggctgacttcggcctgagcaccttccagggcggcagccagagcggcaccggcagcggcgagcccggcggcaccctgggctacctggcccccgagctgttcgtgaatgtgaaccggaaggccagcaccgccagcgacgtgtacagcttcggcatcctgatgtgggccgtgctggccggccgggaggtcgagctgcccaccgagcccagcctggtgtacgaggctgtgtgcaaccggcagaaccggcccagcctggcagagctgccccaggccggccccgagacccccggcctggagggccttaaggagctgatgcagctgtgctggagcagcgagcctaaggaccggcccagcttccaggagtgcctgcccaagaccgacgaggtgttccagatggtcgagaataatatgaacgcagctgtgagcaccgtgaaggacttcctgagccagctgcggagcagcaaccggcggttcagcatccccgagagcggccagggaggcaccgagatggatggcttccggcggaccatcgagaaccagcacagcaggaatgacgtgatggtgagcgagtggctgaacaagctgaacctggaggagccccccagcagcgtgccaaagaagtgccccagcctgaccaagcggagccgggcccaggaggagcaggtgccccaggcctggaccgccggcaccagcagcgacagcatggcccagcccccccagaccccagagacctcaaccttccggaaccagatgcccagccccaccagcaccggcaccccaagccccggcccccggggcaaccagggcgccgagaggcagggcatgaactggagctgccggacccccgagcccaaccccgtgaccggccggcccctggtgaacatctacaactgcagcggcgtgcaggtgggagacaacaactacctgaccatgcagcagaccaccgctctgcccacctggggcctggccccaagcggcaagggccggggcctgcagcaccccccccccgtgggcagccaggagggccccaaggacccagaggcctggagccggccccagggctggtacaaccacagcggcaaggggaccaagtacgagatcgagcggatgctgaaggaactgaaggacctgctggaggagctgaaacggatgctcgaggagctgaagcggagcctgaaggagctgaagaagaacccaagcgaggacgccctggtggagaacaaccggctgatcgtgaaggtgctggaggtgatcgtggaggtgctgcgggccatcatcgagctggccgagctgataatccggagcgactgattaaaccggtctgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgaattcgatatccagcggccgcgctagcgctg

SEQ ID NO: 7 - RNA, 1211 nt, Artificial Sequence
  Description of Artificial Sequence: Synthetic polynucleotide
aggucuucugguccccacagacucagagagaacccgccaccaugggcagcgccuucgagcgcgucguccgccgcgucguccaggagcucgaccacggcggcgaguucauccccgucaccagccuccagagcagcaccggcuuccagcccuacugccucgucguccgcaagcccagcagcagcugguucuggaagccccgcuacaagugcgugaaccugagcaucaaggacauccucgagccggacgccgccgagcccgacguccagcgcggccgcagcuuccacuucuacgacgccauggacggccagauccagggcagcgucgagcuggccgcccccggccaggccaagaucgccggcggcgccgccgucagcgacagcagcagcaccagcaugaacgucuacagccucagcgucgaccccaacaccuggcagacccuccuccacgagcgccaccuccgccagcccgagcacaagguccuccagcagcuccgcagccggggcgacaacgucuacgucgucaccgagguccuccagacccagaaggaggucgaggucacccggacccacaagcgcgagggcagcggccgcuucagccuccccggcgccaccugccuccagggcgagggccagggccaccucagccagaagaagaccgucaccauccccagcggcagcacccucgccuuccgcgucgcccagcucgucaucgacagcgaccucgacguccuccucuuccccgacaagaagcagcgcaccuuccagccccccgccaccggccacaagcgcagcaccagcgagggcgccuggccccagcuccccagcggccucagcaugaugcgcugccuccacaacuuccucaccgacugauuaaaccggucugcagcuuuugccuugguucuuccuagugccuacaaugggaaaacuucacuccaaagagaaaccuauuaagucaucaucuccaaacuaaacccucacaaguaacaguugaagaaaaaauggcaagagaucauauccucagaccagguggaauuacuuaaauuuuaaagccugaaaauuccaauuugggggugggagguggaaaaggaacccaauuuucuuaugaacagauauuuuuaacuuaauggcacaaagucuuagaauauuauuaugugccccguguucccuguucuucguugcugaauucgauauccagcggccgcgcuagcgcug

SEQ ID NO: 8 - RNA, 1214 nt, Artificial Sequence
  Description of Artificial Sequence: Synthetic polynucleotide
aggucuucugguccccacagacucagagagaacccgccaccaugccaagcgccuucgagaagguggugaagaacgugaucaaggaggucuccggcagccggggcgaccugauccccguggacagccugcggaacagcaccagcuuccggcccuacugccugcugaaccggaaguucagcagcagccgguucuggaagccccgguacagcugcgugaaccugagcaucaaggacauccuggagcccagcgcccccgagcccgagccugaguguuucggcagcuucaaggugagcgacgugguggacggcaacauccagggccgggugaugcugagcggcaugggcgagggaaagaucagcgggggcgccgccgugagcgacagcagcagcgccagcaugaacgugugcauccugcgggugacccagaagaccugggagaccaugcagcacgagcggcaccugcagcagccugagaauaagauccugcagcagcugcggagccggggcgaugaccuguucguggugaccgaagugcugcagaccaaggaggaggugcagaucaccgaggugcacagccaggagggcagcggccaguucacccugcccggcgcccugugccugaagggcgagggcaagggacaccagagccggaagaagauggugaccauccccgccggcagcauccuggccuuccggguggcccagcugcugaucggcagcaagugggacauucugcuggugagcgacgagaagcagcggaccuucgagcccagcagcggcgaccggaaggccgugggccagcggcaccacggccugaacgugcuggccgcccuguguagcaucggcaagcagcuaagccugcugagcgacugauuaaaccggucugcagcuuuugccuugguucuuccuagugccuacaaugggaaaacuucacuccaaagagaaaccuauuaagucaucaucuccaaacuaaacccucacaaguaacaguugaagaaaaaauggcaagagaucauauccucagaccagguggaauuacuuaaauuuuaaagccugaaaauuccaauuugggggugggagguggaaaaggaacccaauuuucuuaugaacagauauuuuuaacuuaauggcacaaagucuuagaauauuauuaugugccccguguucccuguucuucguugcugaauucgauauccagcggccgcgcuagcgcug

SEQ ID NO: 9 - RNA, 1670 nt, Artificial Sequence
  Description of Artificial Sequence: Synthetic polynucleotide
aggucuucugguccccacagacucagagagaacccgccaccaugaagugaggcucuccucccgccccgccccucccacgccucaccagccccccgcgcgcccacccuccggcgggugacagcuccgggaucagcaacccuuccugcugcugcuacugcugcugcugcugccgccgccgccgccgccgcugcccuuggguccccccgagucuccgggacugcccucucgacugucaguggggcagccucuccgacucugcacccgccucgaccuccccacccgcucccacaccccugugcccucauguggagccuaagagaacagaacaggccgugaagccagcagagaaaggaagcggagcuacuaacuucagccugcugaagcaggcuggagacguggaggagaacccuggaccuuacucgagcagacgcguagaucugggcuccggcgagggcaggggaagucuucuaacaugcggggacguggaggaaaaucccggcccaggcagcgccuucgagcgcgucguccgccgcgucguccaggagcucgaccacggcggcgaguucauccccgucaccagccuccagagcagcaccggcuuccagcccuacugccucgucguccgcaagcccagcagcagcugguucuggaagccccgcuacaagugcgugaaccugagcaucaaggacauccucgagccggacgccgccgagcccgacguccagcgcggccgcagcuuccacuucuacgacgccauggacggccagauccagggcagcgucgagcuggccgcccccggccaggccaagaucgccggcggcgccgccgucagcgacagcagcagcaccagcaugaacgucuacagccucagcgucgaccccaacaccuggcagacccuccuccacgagcgccaccuccgccagcccgagcacaagguccuccagcagcuccgcagccggggcgacaacgucuacgucgucaccgagguccuccagacccagaaggaggucgaggucacccggacccacaagcgcgagggcagcggccgcuucagccuccccggcgccaccugccuccagggcgagggccagggccaccucagccagaagaagaccgucaccauccccagcggcagcacccucgccuuccgcgucgcccagcucgucaucgacagcgaccucgacguccuccucuuccccgacaagaagcagcgcaccuuccagccccccgccaccggccacaagcgcagcaccagcgagggcgccuggccccagcuccccagcggccucagcaugaugcgcugccuccacaacuuccucaccgacugauuaaaccggucugcagcuuuugccuugguucuuccuagugccuacaaugggaaaacuucacuccaaagagaaaccuauuaagucaucaucuccaaacuaaacccucacaaguaacaguugaagaaaaaauggcaagagaucauauccucagaccagguggaauuacuuaaauuuuaaagccugaaaauuccaauuugggggugggagguggaaaaggaacccaauuuucuuaugaacagauauuuuuaacuuaauggcacaaagucuuagaauauuauuaugugccccguguucccuguucuucguugcugaauucgauauccagcggccgcgcuagcgcug

SEQ ID NO: 10 - RNA, 1673 nt, Artificial Sequence
  Description of Artificial Sequence: Synthetic polynucleotide
aggucuucugguccccacagacucagagagaacccgccaccaugaagugaggcucuccucccgccccgccccucccacgccucaccagccccccgcgcgcccacccuccggcgggugacagcuccgggaucagcaacccuuccugcugcugcuacugcugcugcugcugccgccgccgccgccgccgcugcccuuggguccccccgagucuccgggacugcccucucgacugucaguggggcagccucuccgacucugcacccgccucgaccuccccacccgcucccacaccccugugcccucauguggagccuaagagaacagaacaggccgugaagccagcagagaaaggaagcggagcuacuaacuucagccugcugaagcaggcuggagacguggaggagaacccuggaccuuacucgagcagacgcguagaucugggcuccggcgagggcaggggaagucuucuaacaugcggggacguggaggaaaaucccggcccaccaagcgccuucgagaagguggugaagaacgugaucaaggaggucuccggcagccggggcgaccugauccccguggacagccugcggaacagcaccagcuuccggcccuacugccugcugaaccggaaguucagcagcagccgguucuggaagccccgguacagcugcgugaaccugagcaucaaggacauccuggagcccagcgcccccgagcccgagccugaguguuucggcagcuucaaggugagcgacgugguggacggcaacauccagggccgggugaugcugagcggcaugggcgagggaaagaucagcgggggcgccgccgugagcgacagcagcagcgccagcaugaacgugugcauccugcgggugacccagaagaccugggagaccaugcagcacgagcggcaccugcagcagccugagaauaagauccugcagcagcugcggagccggggcgaugaccuguucguggugaccgaagugcugcagaccaaggaggaggugcagaucaccgaggugcacagccaggagggcagcggccaguucacccugcccggcgcccugugccugaagggcgagggcaagggacaccagagccggaagaagauggugaccauccccgccggcagcauccuggccuuccggguggcccagcugcugaucggcagcaagugggacauucugcuggugagcgacgagaagcagcggaccuucgagcccagcagcggcgaccggaaggccgugggccagcggcaccacggccugaacgugcuggccgcccuguguagcaucggcaagcagcuaagccugcugagcgacugauuaaaccggucugcagcuuuugccuugguucuuccuagugccuacaaugggaaaacuucacuccaaagagaaaccuauuaagucaucaucuccaaacuaaacccucacaaguaacaguugaagaaaaaauggcaagagaucauauccucagaccagguggaauuacuuaaauuuuaaagccugaaaauuccaauuugggggugggagguggaaaaggaacccaauuuucuuaugaacagauauuuuuaacuuaauggcacaaagucuuagaauauuauuaugugccccguguucccuguucuucguugcugaauucgauauccagcggccgcgcuagcgcug

SEQ ID NO: 11-23 - skipped (13 entries)

SEQ ID NO: 24 - DNA, 13 bp, Artificial Sequence
  Description of Artificial Sequence: Synthetic oligonucleotide
  modified_base (2)..(7): a, c, t, g, unknown or other
  modified_base (9)..(10): a, c, t, g, unknown or other
vnnnnnnmnnmwk

SEQ ID NO: 25 - DNA, 13 bp, Artificial Sequence
  Description of Artificial Sequence: Synthetic oligonucleotide
  modified_base (1)..(3): a, c, t, g, unknown or other
  modified_base (6)..(7): a, c, t, g, unknown or other
  modified_base (12)..(12): a, c, t, g, unknown or other
nnnvwnnkghhnh

SEQ ID NO: 26 - DNA, 13 bp, Artificial Sequence
  Description of Artificial Sequence: Synthetic oligonucleotide
  modified_base (5)..(7): a, c, t, g, unknown or other
  modified_base (10)..(12): a, c, t, g, unknown or other
dvhvnnncwnnnb

SEQ ID NO: 27 - DNA, 13 bp, Artificial Sequence
  Description of Artificial Sequence: Synthetic oligonucleotide
  modified_base (4)..(13): a, c, t, g, unknown or other
mwbnnnnnnnnnn

SEQ ID NO: 28 - DNA, 13 bp, Artificial Sequence
  Description of Artificial Sequence: Synthetic oligonucleotide
  modified_base (3)..(4): a, c, t, g, unknown or other
  modified_base (6)..(6): a, c, t, g, unknown or other
  modified_base (8)..(8): a, c, t, g, unknown or other
  modified_base (11)..(13): a, c, t, g, unknown or other
wgnnsnhnhdnnn

SEQ ID NO: 29 - DNA, 13 bp, Artificial Sequence
  Description of Artificial Sequence: Synthetic oligonucleotide
  modified_base (2)..(7): a, c, t, g, unknown or other
  modified_base (9)..(10): a, c, t, g, unknown or other
vnnnnnnmnnmwk

SEQ ID NO: 30 - DNA, 13 bp, Artificial Sequence
  Description of Artificial Sequence: Synthetic oligonucleotide
  modified_base (3)..(4): a, c, t, g, unknown or other
  modified_base (6)..(6): a, c, t, g, unknown or other
  modified_base (8)..(13): a, c, t, g, unknown or other
vmnnwnknnnnnn

(19) United States
(12) Patent Application Publication
(10) Pub. No.: US 2024/0067684 A1
(43) Pub. Date: Feb. 29, 2024

(54) CONSTITUTIVELY ACTIVE PAYLOADS

(71) Applicant: Kernal Biologics, Inc., Cambridge, MA (US)

(72) Inventors: Yusuf Erkul, Cambridge, MA (US); Burak Yilmaz, Cambridge, MA (US); Cafer Ozdemir, Cambridge, MA (US)

(21) Appl. No.: 18/269,258

(22) PCT Filed: Dec. 22, 2021
(86) PCT No.: PCT/US21/64934

§ 371 Date: Jun. 22, 2023

Related U.S. Application Data
(60) Provisional application No. 63/130,207, filed on Dec. 23, 2020.

(51) Int. Cl. C07K 14/47; A61K 47/69; A61P 35/00

(52) U.S. Cl. CPC C07K 14/4705; A61K 47/6929; A61P 35/00

(57) ABSTRACT

The present disclosure provides, among other things, methods and compositions useful for induction of cell death. The present disclosure provides translatable nucleic acids encoding constitutively active payloads capable of inducing cell death. Payloads of the present disclosure are particularly useful in induction of immunogenic cell death.

### CROSS REFERENCE TO RELATED APPLICATIONS

This application claims priority to U.S. Provisional Patent Application No. 63/130,207 filed Dec. 23, 2020 the entire contents of which is hereby incorporated by reference

### BACKGROUND

There is a need to develop improved therapies for the treatment of cellular disease (e.g., cancer). Therapies designed to induce cell death and generate an immune response provide an opportunity for unique treatment options.

### SUMMARY

The present disclosure provides technologies that achieve killing of target cells. Among other things, the present disclosure provides technologies that achieve such killing through delivery of translatable RNA molecules.

Delivery of nucleic acids for therapeutic purposes is a burgeoning and powerful field. Significant progress has recently been made in the field, including specifically with respect to technologies for stabilizing and/or effecting delivery of nucleic acids, particularly including translatable RNA molecules (e.g., mRNA).

Among other things, the present disclosure provides an insight that harnessing translatable nucleic acid (e.g., RNA) technologies to achieve expression of an encoded cell death polypeptide could offer unique utilities and advantages. The present disclosure provides, among other things, translatable nucleic acid constructs (e.g., mRNAs) that encode constitutively active cell death polypeptides.

Furthermore, the present disclosure provides an insight that incorporation of certain features into provided translatable nucleic acid constructs, for example that limit expression of protein encoded by such constructs to particular cell types and/or physiological states can provide unique and powerful therapeutic applications.

The present disclosure provides an insight that controlled expression constructs can be developed that achieve effective expression of cell death polypeptide payloads. Moreover, the present disclosure provides constructs that achieve expression of cell death polypeptides, including, in some embodiments, that achieve specific expression under certain conditions and/or in certain cell types.

In some embodiments, the present disclosure provides a method of treating a subject, wherein the method comprises administering a therapeutically effective amount of an in-vitro transcribed mRNA encoding a constitutively active polypeptide. In some embodiments, the present disclosure provides a pharmaceutical composition comprising an in-vitro transcribed mRNA encoding a constitutively active polypeptide. In some embodiments, the present disclosure provides an in-vitro transcribed mRNA encoding a constitutively active polypeptide.

### BRIEF DESCRIPTION OF THE DRAWING

FIG. 1 shows an exemplary construct of a nucleic acid as described in the present disclosure. A synthetic mRNA, in some embodiments, comprises a 5′ cap, a start codon (AUG), a constitutively active (c.a.) suicide protein (e.g., gasdermin (GasD) a stop codon, and a poly-A tail.

FIGS. 2A-2C demonstrate induction of immunogenic cell death by in vitro transcribed mRNAs constitutively active suicide proteins.

FIGS. 3A-3D demonstrates dose responsive induction of immunogenic cell death by in vitro transcribed mRNAs encoding constitutively active suicide proteins.

FIG. 4 shows an exemplary construct of a nucleic acid as described in the present disclosure comprising an oncoselective readthrough element (RT Element). A synthetic mRNA, in some embodiments, comprises a 5′ cap, an oncoselective readthrough element, a linker (e.g., PT2A), a constitutively active (c.a.) suicide protein (e.g., gasdermin (GasD) a stop codon, and a poly-A tail.

FIGS. 5A-5B demonstrate dose responsive oncoselective induction of cell death.

FIG. 6 shows proteins and pathways involved in immunogenic cell death.

FIG. 7 demonstrates induction of tumor cell death by constitutively active RIPK3 encoded by mRNA

FIGS. 8A and 8B demonstrate reduction in tumor volume by treatment with mRNA encoding constitutively active RIPK3.

### DEFINITIONS

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumoral, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: As used herein, the term “amino acid” refers to any entity that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva. In some embodiments, a cancer may be or comprise one or more solid tumors. In some embodiments, a cancer may be or comprise one or more haematologic tumors.

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc. that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at position 190 in the reference polypeptide; those of ordinary skill in the art readily appreciate how to identify “corresponding” amino acids (see. e.g., Benson et al. Nucl. Acids Res. (1 Jan. 2013) 41 (D1): D36-D42; Pearson et al. PNAS Vol.85, pp. 2444-2448, April 1988). Those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Expression: As used herein, the term “expression” of a nucleic acid sequence refers to the generation of a gene product from the nucleic acid sequence. In some embodiments, a gene product can be a transcript (e.g., a primary transcript or a processed transcript such as an mRNA). In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanking Sequence: As used herein the term “flanking sequence” refers to any sequence that precedes or succeeds a sequence or domain of interest. For example, a region upstream of a stop codon can be referred to as “upstream flanking region”.

Gene: As used herein, the term “gene” refers to a DNA or RNA sequence that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression). In some embodiments, a gene is located or found (or has a nucleotide sequence identical to that located or found) in a genome (e.g., in or on a chromosome or other replicable nucleic acid).

in vitro transcribed mRNA: As used herein, the term “in vitro transcribed mRNA” refers to an mRNA that is synthesized using a DNA template and an RNA polymerase enzyme in a cell-free environment. In some embodiments, an RNA polymerase comprises a viral RNA polymerase enzyme such as T7 RNA polymerase or SP6 RNA polymerase. In some embodiments, a DNA template comprises a plasmid DNA that is linearized using restriction enzymes. In other embodiments, a DNA template is a linear PCR product.

Mutant: As used herein, the term “mutant” refers to an organism, a cell, or a biomolecule (e.g., a nucleic acid or a polypeptide) that has a genetic variation as compared to a reference organism, cell, or biomolecule. For example, a mutant nucleic acid or polypeptide may, in some embodiments, have, for example, a substitution of one or more residues (e.g., of one or more nucleobases or amino acids), a deletion of one or more residues (e.g., an internal deletion or a truncation), an insertion of one or more residues, an inversion of two or more residues, etc, as compared to a reference nucleic acid molecule. Those skilled in the art will be familiar with various particular types of such nucleic acid or polypeptide mutants—e.g., fusions, indels, etc. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of activity (e.g., of binding activity, enzymatic activity, etc), or a partial loss of function, e.g., a diminished activity (e.g., binding activity, enzymatic activity, etc). In some embodiments, a mutant comprises a genetic variant that is associated with a gain of function, e.g., with enhancement of an existing activity, or gain of a new activity relative to an appropriate reference (e.g., the same entity absent the genetic variation). In some embodiments, a gain of function mutant may have gained an alteration in a characteristic or activity. In some embodiments, a gain of function mutant may have constitutive activity. In some embodiments, a loss of function mutant may have lost (or reduced relative to a reference) a desirable activity. In some embodiments, the reference organism, cell, or biomolecule relative to which a mutant's structure, level, and/or activity is compared, is a wild-type organism, cell, or biomolecule.

Nucleic acid: As used herein, the term “nucleic acid” refers to a polymer of at least three nucleotides. In some embodiments, a nucleic acid is or comprises DNA. In some embodiments, a nucleic acid is or comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Peptide: As used herein, the term “peptide” refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and/or substantially pyrogen-free.

Polypeptide: As used herein, the term “polypeptide”, refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” refers to a standard or control relative to which a comparison is performed. For example, in some embodiments, an agent, animal, individual, population, sample, sequence, or value of interest is compared to a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a source of interest, as described herein. In some embodiments, a source of interest is or comprises an organism, such as a microbe, a plant, an animal or a human. In some embodiments, a biological sample is or comprises biological tissue or fluid, or one or more components thereof. In some embodiments, a biological sample may be or comprise bone marrow; blood; blood cells; ascites; tissue or biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a biological sample comprises cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Therapeutic agent: As used herein, the term “therapeutic agent” generally refers to an agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, therapeutic agents may be CREBBP antagonists as described herein.

Therapeutically effective amount: As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Upstream and downstream: As used herein when describing RNA the term “upstream” refers to toward or close to the 5′ end of the RNA molecule and the term “downstream” refers to toward or close to the 3′ end” of the RNA molecule. As used herein when describing DNA, “upstream “is toward the 5′ end of the coding strand and “downstream” is toward the 3′ end of the coding strand. Because of the anti-parallel orientation of DNA, this means the 3′ end of the template strand is upstream and the 5′ end is downstream.

Variant As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### Translatable Nucleic Acid

One of skill in the art will appreciate that a variety of technologies are available for generating a nucleic acid that is translatable by mammalian polypeptide expressions systems. One of skill will also recognize that expression of a polypeptide in a mammalian system is a highly regulated process. One of skill will recognize that not all nucleic acids that encode a polypeptide are equally effective in expressing a polypeptide or inducing a desired effect in a mammalian cell. In some embodiments, expression of a polypeptide from a DNA in a mammalian cell induces a desired effect but expression of the same polypeptide from an RNA does not.

In some embodiments, the present disclosure provides a translatable nucleic acid construct. In some embodiments, a translatable nucleic acid is an RNA. In some embodiments, a translatable nucleic acid is an mRNA. In some embodiments a translatable nucleic acid is an in-vitro transcribed mRNA. In some embodiments, the present disclosure recognizes that an RNA has certain advantages over DNA (e.g., polypeptide encoded by RNA (e.g., mRNA) more readily expressed in mammalian systems). In some embodiments a translatable nucleic acid is not a DNA.

In some embodiments, a translatable nucleic acid comprises a translation regulatory element. In some embodiments a translation regulatory element comprises a 5′UTR. In some embodiments a translation regulatory element comprises a 5′ cap (e.g., 5′ 7-methylguanylate cap). In some embodiments, a translatable nucleic acid encodes a poly A tail. In some embodiments, a translatable nucleic acid comprise and internal ribosomal entry site.

In some embodiments a translation regulatory element comprises a ribosome stop codon read-through element. In some embodiments an oncoselective readthrough element is a structural feature as described in International Patent Application No. PCT/US 20138742 the contents of which are incorporated herein by reference.

Without wishing to be bound by any particular theory, the present disclosure observes that ribosome read-through of stop codons can be caused by interactions between the 18s rRNA and an RNA (e.g., an mRNA) bound by the ribosome. For example, helices of the rRNA may interact with mRNA sequences. See Namy et al. EMBO Rep. 2001 Sep. 15; 2(9): 787-793 describing interactions of helix 17 of rRNA in S. cervisae with mRNA bound by the ribosome that leads to stop codon read-through. The present disclosure recognizes, among other things, that human rRNA helix 37 can interact with sequences of mRNA that contribute to stop codon read-through.

Alternatively or additionally, oncoselective ribosome stop codon read-through can be induced and/or enhanced by including one or more particular structural features in a translatable nucleic acid (e.g., an RNA such as an mRNA). In some embodiments, one or more primary structure features of a translatable nucleic acid (e.g., an RNA such as an mRNA) can be used to induce and/or enhance oncoselective stop codon read-through. Alternatively or additionally, in some embodiments, one or more secondary and/or tertiary structure features (e.g. stem loop, bulge loop, kissing loop, pseudoknots, or branch loop) of a translatable nucleic acid (e.g., an RNA such as an mRNA) can be used to induce and/or enhance oncoselective stop codon read-through. In some embodiments, a structural feature capable of inducing and/or enhancing stop codon read-through is within the first 50, 60, 70, 80, 90, 100, 110, or 120 nucleotides of the downstream flanking sequence. In some further embodiments, portions of a structural feature capable of inducing and/or enhancing stop codon read-through is comprised by the first 16 nucleotides of the downstream flanking sequence.

In some embodiments, a structural feature capable of inducing and/or enhancing stop codon read-through comprises 10, 20, 30, 40, 50 or more base paired nucleotides within the first 10, 20, 30, 40, 50, 60 or more nucleotides of the downstream flanking sequence.

In accordance with some embodiments of the present disclosure, stop codon read-through can be induced and/or enhanced through use of oncoselective read-through motifs as described herein.

Alternatively or additionally, in some embodiments, inclusion of one or more regions of high G-C content can be used to induce oncospecific stop codon read-through. For example, in some embodiments, high G-C content in the 3′UTR of a translatable nucleic acid (e.g., of an RNA such as an mRNA) can be used to induce and/or enhance oncospecific stop codon read through. In some embodiments, high G-C content in the nucleotides preceding a stop codon can be used to induce and/or enhance oncospecific stop codon read-through of that stop codon. In some embodiments, high G-C content in the 60 nucleotides preceding a stop codon can be used to induce and/or enhance oncospecific stop codon read-through of that stop codon. In some embodiments, high G-C content in 50 nucleotides following a stop codon can be used to induce and/or enhance oncospecific stop codon readthrough of that stop codon. In some embodiments, high G-C content in the first 120 nucleotides after a stop codon (i.e., in the 3′UTR) can be used to induce and/or enhance oncospecific stop codon readthrough of that stop codon. In some embodiments, high G-C content means a log-odds of binomial probability of 4 or greater relative to a non-readthrough transcript. In some embodiments, a readthrough motif comprises GC content of more than 42%, more than 48%, preferably more than 54% in the downstream flanking sequence.

In some embodiments, the readthrough motif comprises the amino acid sequence VNNNNNNMNNMWK (SEQ ID NO. 24), NNNVWNNKGHHNH (SEQ ID NO. 25), DVHVNNNCWNNNB (SEQ ID NO. 26), MWBNNNNNNNNNN (SEQ ID NO. 27), WGNNSNHNHDNNN (SEQ ID NO. 28), VNNNNNNNMNNMWK(SEQ ID NO. 29) or VMNNWNKNNNNNN (SEQ ID NO. 30), wherein V stands for A, C or G, M stands for A or C, W stands for A or T/U, K stands for G or T/U, H stands for A, C or T/U, D stands for A, G or T/U, B stands for C, G or T/U, S stands for G or C, N stands for any nucleotide, within the region that spans the readthrough stop codon and the first 14 nucleotides of the downstream flanking sequence.

The present disclosure further provides an insight that inclusion of a codon resulting in introduction of proline to the nascent polypeptide can induce kinking of the nascent polypeptide, and that such kinking can be used to induce and/or enhance oncoselective stop codon read-through. Thus, in some embodiments, oncoselective stop-codon read through can be induced and/or enhanced by inclusion of one or more proline-encoding codons in a translatable nucleic acid, as an alternative to or in addition to one or more of the other strategies described herein for inducing and/or enhancing oncoselective stop codon read-through.

In some embodiments, a stem loop in the mRNA can induce and/or enhance stop codon read-through. In some embodiments, a stem loop inducing and/or enhancing stop codon readthrough is within approximately 20, 40, 60, 80 or 120 nucleotides of the stop codon. In some embodiments, a stem loop inducing and/or enhancing stop codon read-through is in the coding sequence just prior to the stop codon. In some embodiments, a stem loop inducing and/or enhancing stop codon read-through is in the 3′UTR. In some embodiments, a stem loop inducing and/or enhancing stop codon read-through is in the region spanning the coding region and 3′UTR boundary. In some embodiments, a bulge loop or a pseudoknot in the mRNA can induce and/or enhance stop codon read-through. In some embodiments, nucleic acid structures inducing and/or enhancing stop codon read-through have a low Gibbs free energy relative to nucleic acid structures that do not result in read-through. In some embodiments, the first 25, 50, or 75 nucleotides of the 3′UTR of a nucleic acid inducing stop codon read-through have a delta G of 5 kcal/mole;10 kcal/mole; 15 kcal/mole; 20 kcal/mole; 25 kcal/mole; 30 kcal/mole lower than non-cancer stop codon read-through counterparts. In some embodiments, the first 25, 50, or 75 nucleotides of the 3′UTR of a nucleic acid inducing stop codon read-through have a delta G in the range of 5 kcal/mole to 20 kcal/mole; 5 kcal/mole to 10 kcal/mole; or 10 kcal/mole to 20 kcal/mole; 25 kcal/mole; 30 kcal/mole lower than non-cancer stop codon read-through counterparts.

In some embodiments, aminoglycosides (e.g., gentamicin) and macrolides (e.g. erythromycin) can induce stop codon read-through. Without wishing to be bound by any theory, aminoglycosides can induce stop codon read-through by binding 18 s rRNA and macrolides can induce stop codon read-through by binding the peptide channel within large ribosomal subunit. In some embodiments, aminoglycosides and macrolides can induce stop codon read-through in healthy (normal) cells. In some embodiments, subjects treated with aminoglycosides or macrolides should not be treated with a nucleic acid comprising a stop codon read-through motif

In some embodiments, the present disclosure encompasses the recognition that an oncoselective translation sequence element can be oncospecific and result in translation and payload expression only in cancer cells (i.e., no detectable expression in non-cancer cells). Alternatively or additionally, in some embodiments, an oncoselective translation sequence element is translated 2, 5, 10, 15, 20, 30 or more—fold higher in cancer cell(s) as compared with appropriately comparable non-cancer cells.

In some embodiments, an oncoselective translation sequence element can comprise an internal ribosome entry segment/site (IRES). In some embodiments, an oncogenic ribosome, or RNA binding protein, preferentially binds an IRES in an oncoselective translation sequence element. In some embodiments, an oncoselective translation sequence element can be bound by or direct the binding of translation initiating RNA binding proteins (RBPs). In some embodiments, an oncoselective translation sequence element can comprise and IRES and be bound by or direct the binding of RBPs.

In some embodiments a linker may be present between an oncoselective translation sequence element and a sequence encoding a polypeptide (e.g. a protein that induces cell death). In some embodiments, a linker comprises 2A linker. In some embodiments, a linker comprises a PT2A linker. In some embodiments, a linker comprises a F2Am linker.

### Cell Death

In some embodiments, a translatable nucleic acid encodes a polypeptide that induces cell death. In some embodiments a translatable nucleic acid encodes a polypeptide that induces, immunogenic cell death, for example, necroptosis, pyroptosis or ferroptosis. In some embodiments a translatable nucleic acid encodes a component of a cell death pathway. See, for example, Tang et al., Cell Research volume 29, pages 347-364(2019) and Galluzi et al., Cell Death & Differentiation volume 25, pages 486-541(2018). In some embodiments a translatable nucleic acid encodes a suicide protein.

### Suicide Proteins

Those skilled in the art will be aware of various proteins commonly referred to as “suicide proteins” (encoded by “suicide genes”) and will appreciate that, in some embodiments, a protein encoded by a translatable nucleic acid as described herein (e.g., an in vitro transcribed mRNA) is or comprises a suicide protein.

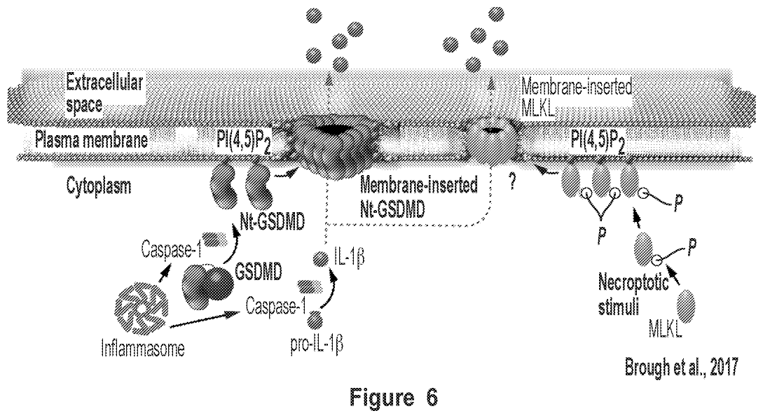

In some embodiments, a suicide protein is a protein that induces cell death. In some embodiments, a suicide protein is a protein that induces immunogenic cell death, such as necroptosis, pyroptosis or ferroptosis (See FIG. 6). The present disclosure provides an insight that certain suicide proteins that induce necroptosis may be particularly advantageous for use in accordance with the present disclosure. For example, the present disclosure observes that necroptosis can induce and/or promote an adaptive immune response. Without wishing to be bound by any particular theory, the present disclosure observes that necroptosis involves immune ligands including Fas, TNF, and LPS leading to activation of RIPK. Dhuriya and Sharma J Neuroinflammation. 2018 Jul. 6;15(1):199; Linkermann and Green N Engl J Med. 2014 Jan. 30; 370(5): 455-465. The present disclosure teaches that use of a necroptotic suicide protein, which may induce and/or promote an adaptive immune response, may facilitate inhibition, destruction and/or removal of tumor cells. In some embodiments, a suicide protein induces apoptosis; in some such embodiments, a suicide protein is p53, or is a protein involved in a p53-mediated apoptosis pathway (e.g. PUMA, BIM, BAX, BAK, tBID, CASPASE-3, CASPASE-7, CASPASE-8, CASPASE-9).

In some embodiments, a suicide protein is or comprises a protein that renders cells expressing it more susceptible to killing by a separate agent. To give but one example, those skilled in the art are aware of certain viral and/or bacterial enzymes that are not naturally found in mammals and that convert a substance that may be harmless to cells that do not express the enzyme(s) into a toxin. In some embodiments, such a suicide protein is or comprises an enzyme that converts an otherwise inactive agent (e.g., drug) into a toxic antimetabolite, e.g., that inhibits nucleic acid synthesis. In some such embodiments, a suicide protein is a thymidine kinase, wherein the payload sequence encoding thymidine kinase is co-administered with or administered before ganciclovir or valacyclovir treatment.

In some embodiments, a suicide protein payload for use in accordance with the present disclosure is Mixed Lineage Kinase Domain Like Pseudokinase (MLKL), Receptor-interacting serine/threonine-protein kinase 3 (RIPK3), Receptor-interacting serine/threonine-protein kinase 1 (RIPK1), Fas-associated protein with death domain (FADD), or gasdermin D (GSDMD), cysteine-aspartic proteases, cysteine aspartases or cysteine-dependent aspartate-directed proteases (CASPASE-1 or CASP-1), CASPASE-4, CASPASE-5, CASPASE-12, PYCARD/ASC (PYD and CARD domain containing/Fas-associated protein with death domain) or variants thereof.

### Constitutively Active Polypeptides

In some embodiments, the present disclosure recognizes that expression of a suicide protein with constitutive activity is advantageous. In some embodiments, the present disclosure provides a translatable nucleic acid that encodes a constitutively active polypeptide. In some embodiments, the present disclosure provides a translatable nucleic acid that encodes a constitutively active suicide protein.

In some embodiments, a constitutively active polypeptide is a polypeptide whose activity is not regulated by post translational modifications (e.g., phosphorylation, glycosylation, methylation, lipidation, etc.). In some embodiments, a constitutively active polypeptide comprises a gain of function mutation relative to the wild type amino acid sequence. In some embodiments, a constitutively active polypeptide has activity in the absence of a substrate or binding partner (e.g., ligand) required by a an appropriate corresponding reference polypeptide (e.g., a wild type protein).

### Delivery

Those skilled in the art, reading the present disclosure, will appreciate that a variety of technologies are available to achieve delivery of a translatable nucleic acid to cells (e.g., cancer cells) in accordance with the present disclosure, and furthermore will appreciate that some modes of delivery involve administration of a composition comprising the translatable nucleic acid (e.g., mRNA).

### Nanoparticle Delivery

As noted herein, those skilled in the art will be aware that a variety of administration systems have been developed to achieve effective delivery of nucleic acids into cells, including within mammalian (e.g., human) subjects.

Among such available technologies are various nanoparticle technologies including, for example, hydrogel, lipid, and/or polymer nanoparticle technologies.

In some embodiments, a nucleic acid is delivered to a subject in accordance with the present disclosure using a lipid nanoparticle. As used herein, the phrase “lipid nanoparticle” refers to a transfer vehicle comprising one or more lipids (e.g., cationic lipids, non-cationic lipids, and PEG-modified lipids). In some embodiments, lipid nanoparticles are formulated to deliver one or more copies of the nucleic acid to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

In some embodiments, a nucleic acid is delivered to a subject in accordance with the present disclosure using a polymer nanoparticle. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

In some embodiments, lipids for use in the delivery of a nucleic acid of the present invention include those described in international patent publication WO 2010/053572, incorporated herein by reference. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g, (15Z, 18Z)-N,N-dimethyl-6-(9Z, 12Z)-octadeca-9,12-dien-1-yl)tetracosa-15,18-dien-1-amine (HGT5000), (15Z, 18Z)-N,N-dimethyl-6-((9Z, 12Z)-octadeca-9,12-dien-1-yl)tetracosa-4,15,18-trien-1-amine (HGT5001), and (15Z,18Z)-N,N-dimethyl-6-((9Z, 12Z)-octadeca-9, 12-dien-1-yl)tetracosa-5,15,18-trien-1-amine (HGT5002).

In some embodiments, the lipid N-(2,3-dioleyloxy)propyll-N,N,N-trimethylammonium chloride or “DOTMA” is used. (Feigner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”,1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”,1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-DMA”, 2,2-dilinoleyl-4-dimethylaminoethyl[1,3]-dioxolane or “DLin-K-XTC2-DMA”, and 2-(2,2-di((9Z,12Z)-octadeca-9,1 2-dien-1-yl)-1,3-dioxolan-4-yl)-N,N-dimethylethanamine (DLin-KC2-DMA)) (See, WO 2010/042877; Semple et al., Nature Biotech. 28: 172-176 (2010) (Heyes, J., et al., J Controlled Release 107: 276-287 (2005); Morrissey, D V., et al., Nat. Biotechnol. 23(8): 1003-1007 (2005); PCT Publication WO2005/121348A1.), DLin-MC3-DMA (See WO2015199952A1 Tam and Cullis Pharmaceutics. 2013 September; 5(3): 498-507) or mixtures thereof. The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Choi (N,N-dimethyl-N-ethylcarboxamidocholesterol),1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

### Subjects

As described herein, the present disclosure provides technologies that are particularly useful in inducing the death of mammalian cells. In some embodiments, the present disclosure provides technologies that are particularly useful in removal of diseased cells from a subject. In some embodiments, the present disclosure provides technologies that are particularly useful in treatment of cancer, precancerous/premalignant conditions (e.g. clonal hematopoiesis of indeterminate potential, carcinoma in situ, leukoplakia), autoimmune lymphoproliferative syndrome, ataxia telangiectasia, xeroderma pigmentosum, polycythemia vera, and familial hemophagocytic lymphohistiocytosis.

In some embodiments, provided technologies are applied to subjects suffering from cancer. That is, in some embodiments, a translatable nucleic acid as described herein (e.g., an in-vitro transcribed mRNA encoding a constitutively active protein) is delivered to (e.g., by administration of a composition comprising the translatable nucleic acid) a subject suffering from cancer.

In some embodiments, a subject has received, is receiving and/or will receive other therapy (e.g., other therapy to treat the cancer and/or one or more side effects of the cancer or its treatment). In some such embodiments, a composition comprises a protein that increases susceptibility of cells to the other therapy.

### Uses

In some embodiments, the present disclosure provides methods of killing cells. In some embodiments, the present disclosure provides methods of inducing cell death. One of skill in the art will recognize many uses for induction of cell death by the compositions and methods of the present disclosure. In some embodiments, a translatable nucleic acid of the present disclosure is used in method of treating cancer.

### Cancer

The present disclosure provides, among other things, methods and compositions useful in the treatment of cancer, e.g., for the treatment of a tumor in a subject.

Cancer is among the leading causes of death worldwide; the number of new cancer cases diagnosed per year is expected to exceed 23 million by 2030. According to statistics released by the United States National Cancer Institute, in 2018, more than 1.7 million new cases of cancer were diagnosed in the United States, and more than 600 thousand people died from the disease.

The most common cancers, in descending order, are breast cancer, lung and bronchus cancer, prostate cancer, colon and rectum cancer, melanoma of the skin, bladder cancer, non-Hodgkin lymphoma, kidney and renal pelvis cancer, endometrial cancer, leukemia, pancreatic cancer, thyroid cancer, and liver cancer. More than 35% of men and women are expected to be diagnosed with cancer at some point during their lifetimes.

In some embodiments, a tumor or cancer suitable for treatment in accordance with the present disclosure includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma , Atypical Rhabdoid Tumor, Basal Cell Carcinoma, Bile Duct Cancer, Bladder Cancer, Bone Cancer , Brain Tumor, Breast Cancer, Bronchial Tumor, Burkitt Lymphoma, Carcinoid Tumor , Carcinoma, Cardiac (Heart) Tumor, Central Nervous System Tumor , Cervical Cancer, Cholangiocarcinoma, Chordoma, Chronic Lymphocytic Leukemia (CLL), Chronic Myelogenous Leukemia (CML), Chronic Myeloproliferative Neoplasm, Colorectal Cancer, Craniopharyngioma, Cutaneous T-Cell Lymphoma, Ductal Carcinoma In Situ (DCIS), Embryonal Tumor , Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Tumor , Kaposi Sarcoma, Kidney Tumor, Langerhans Cell Histiocytosis , Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome , Myelodysplastic/Myeloproliferative Neoplasm, Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer , Renal Cell (Kidney) Cancer, Retinoblastoma, Retinoblastoma, Rhabdomyosarcoma, Rhabdomyosarcoma, Salivary Gland Cancer, Sarcoma, Sézary Syndrome, Skin Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenstrom Macroglobulinemia, Wilms' Tumor. In some preferred embodiments, a tumor or cancer suitable for treatment in accordance with the present disclosure comprises cancers with high frequency of p53 mutation or inactivation, including lung cancer (both non-small cell lung cancer and small cell lung cancer), colon cancer, pancreatic cancer, head and neck cancer, esophageal cancer, ovarian cancer (e.g. high-grade serous ovarian cancer), bladder cancer, liver cancer, gastric cancer, melanoma, AML (e.g. therapy related AML, complex Karyotype AML, AML with 17p deletion), chronic myeloid leukemia, and Burkitt's lymphoma.

### EXEMPLIFICATION

### Example 1 Induction of Immunogenic Cell Death by Translatable Nucleic Acid Constructs

The present example demonstrates the effective killing of mammalian cells by translatable nucleic acids. Specifically, the present example demonstrates introduction of an in vitro transcribed mRNA encoding a constitutively active suicide protein into mammalian cell results in immunogenic cell death. It is understood in the art, and confirmed in the present Example, that expression of a polypeptide encoded by an administered DNA may have different characteristics or features that are observed for expression of the same polypeptides expressed from an administered RNA. Happily, the present disclosure demonstrates effective expression achieved from administered RNA constructs, and furthermore demonstrates particularly desirable features of such expression and/or of certain such constructs.

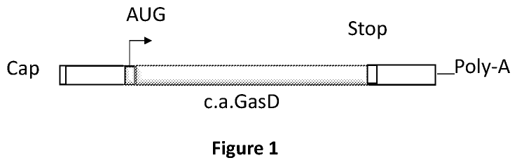

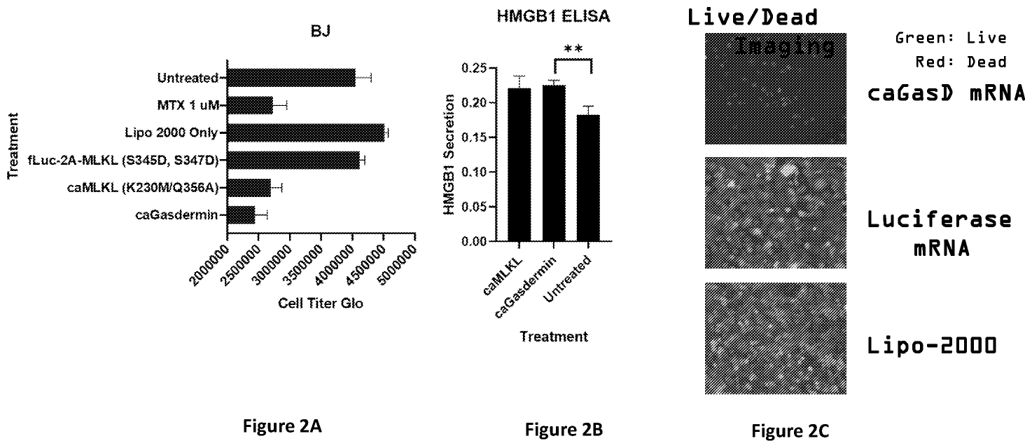

To assess the ability of a constitutively active suicide protein expressed from an mRNA to induce cell death several in vitro transcribed mRNAs encoding various constitutively active suicide proteins were constructed. FIG. 1 provides an exemplary in vitro transcribed mRNA. FIG. 2A demonstrates that introduction of some in vitro transcribed mRNAs results in induction of cell death while others do not. Specifically, an in vitro transcribed mRNA fLUC-2A-MLKL comprising mutations S345D, and S347D (SEQ ID NO. 4), was unable to induce cell killing. This is contrary to the findings of Rodriguez et al. that expression of this mutated MLKL from a DNA construct resulted in expression of a constitutively active MLKL (Cell Death Differ. 2016 January; 23(1): 76-88). FIG. 2A, however, does demonstrate that an in vitro transcribed mRNA encoding a constitutively active MLKL comprising mutations K230M and Q356A (See Yoon et. al. Cell Death Differ. 2016 February; 23(2): 253-260; SEQ ID NO. 2) is capable of inducing cell death. Additionally, a construct encoding for the N-terminal domain of gasderminD (caGasdermin; see, for example, Sborgi et al., EMBO J. 2016 Aug. 15; 35(16): 1766-1778; SEQ ID NO. 3) was capable of inducing cell death.

Further, FIG. 2B shows that mRNA constructs encoding constitutively active MLKL and constitutively active Gasdermin are capable of inducing HMGB1 release from cells. HMGB1 is released from dying cells and participates in stimulation of an immunogenic response to cell death (see, for example, Serrano-del Valle et al. Front. Cell Dev. Biol., 16 Apr. 2019). FIG. 2C shows that an mRNA construct encoding constitutively active Gasdermin effectively killed cells in culture.

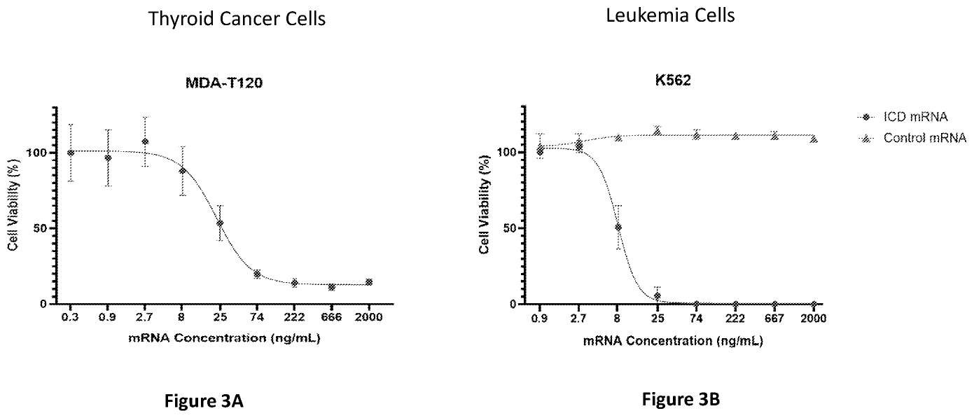

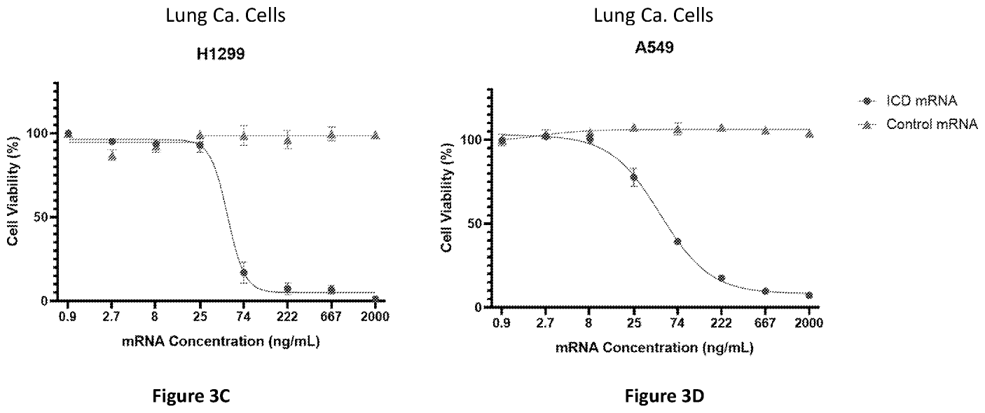

FIG. 3 demonstrates dose responsive induction of immunogenic cell death by in vitro transcribed mRNAs encoding constitutively active gasdermin. Introduction of a mRNA construct encoding constitutively active gasdermin was able to induce cell death in a dose responsive manner in thyroid cancer cells (FIG. 3A); leukemia cells (FIG. 3B); and lung cancer cells (FIGS. 3C and 3D).

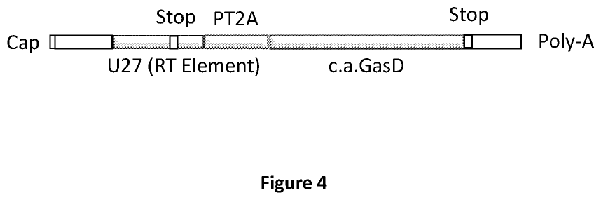

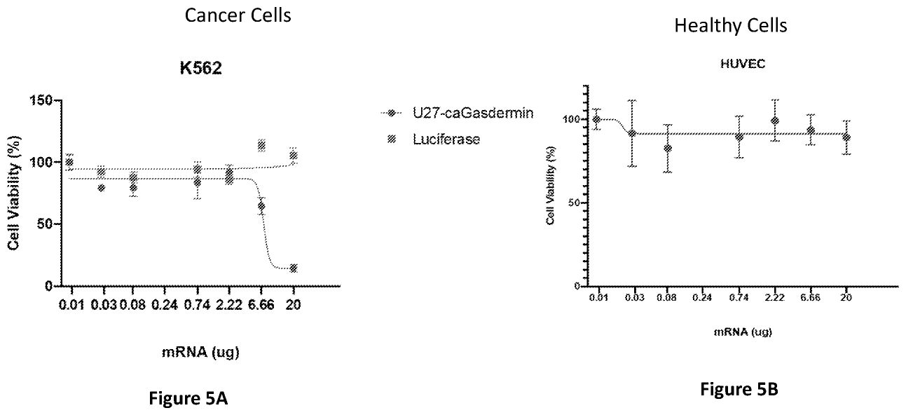

FIG. 5 demonstrates cancer specific dose responsive killing by an mRNA construct comprising a stop codon readthrough element (U27) an example of which is shown in FIG. 4. Introduction of an mRNA construct comprising a stop codon readthrough element (U27) and encoding constitutively active gasdermin (SEQ ID NO. 1) selectively kills cancer cells (FIG. 5A) and does not kill healthy cells (FIG. 5B). Thus, not only are the mRNA constructs of the present disclosure particularly useful at induction of cell death, including immunogenic cell death, when linked with a stop codon readthrough element they are also selective killers of cancer cells.

### Example 2: Additional Translatable Nucleic Acid Constructs for the Induction of Immunogenic Cell Death

The present example provides additional components of cell death pathways that can be included in translatable nucleic acid constructs to induce immunogenic cell death. In vitro transcribed mRNA constructs are designed that encode constitutively active versions of Mixed Lineage Kinase Domain Like Pseudokinase (MLKL), Receptor-interacting serine/threonine-protein kinase 3 (RIPK3), Receptor-interacting serine/threonine-protein kinase 1 (RIPK1), Fas-associated protein with death domain (FADD), or gasdermin D (GSDMD), cysteine-aspartic proteases, cysteine aspartases or cysteine-dependent aspartate-directed proteases (CASPASE-1 or CASP-1), CASPASE-4, CASPASE-5, CASPASE-12, PYCARD/ASC (PYD and CARD domain containing / Fas-associated protein with death domain) or variants or fragments thereof. The in vitro transcribed mRNA constructs are introduced into mammalian cells (e.g., cancer cells) and evaluated for their ability to induce cell death (e.g., immunogenic cell death).

### Example 3: Materials and Methods

The present example provides a description of materials and methods used for the data described herein.

LNP mRNA Production

In vitro Transcription (IVT) of mRNA unmodified mRNAs were transcribed from DNA templates with HiScribe™ T7 High Yield RNA Synthesis Kit using manufacturer's protocols. IVT reaction was run at 37° C. for 2 hours. Reaction product was treated with TURBO DNase (Thermo Fisher) at 37° C. for 10 minutes and mRNA was isolated with a MEGAclear Transcription Clean-Up kit (Thermo Fisher). Capping was performed post-transcriptionally using Vaccinia Capping System (NEB) and mRNA Cap 2′-O-Methyltransferase (NEB). Phosphatase treatment was carried out with Antarctic Phosphatase enzyme (NEB) followed by isolation with MEGAclear Transcription Clean-Up Kit. Capped and dephosphorylated mRNA was spin column purified. RNA concentration was measured with NanoDrop 2000 UV-Vis spectrophotometer (Thermo Fisher). To formulate mRNAs, Genvoy ILM kit (Precision Nanosystems) was used according to user manual using N:P ratio of 6 and flow rate ratio of 3:1. Formulation were prepared using PNI Benchtop or Ignite systems. Downstream processing was carried out with 100 kDa Amicon filters. Each well was treated with 200 ng mRNA LNPs preincubated at 37° C. with 0.1 ul of (1 mg/ml) ApoE.

### HMGB1 ELISA

LL/2 cells (ATCC, CRL-1642) were seeded in triplicates on a 96-well plate at 30,000 cells/well density. 24 hours later cells were transfected with mRNAs. Extracellular HMGB1 secretion was measured the following day on cell culture supernatants via HMGB1 ELISA kit (TECAN, ST51011) according to manufacturer's protocol for sensitive method. ELISA plates were measured on SpectraMax i3x plate reader (Molecular Devices).

### Cell Viability

Cells are seeded on a clear bottom 96-well plate at 30,000 cells/well density. The next day, the cells are transfected with the mRNA constructs. After 24 hrs of transfection, LIVE/DEAD Viability Kit (ThermoFisher Scientific L3224) is administered to the cell culture media and the cells are imaged in a fluorescent microscope. In another plate, cells are administered 100u1 of Cell Titer Glo reagent (Promega, G7571) per well is administered. Luminescence is measured on a GloMax plate reader.

### Does Response Curves:

Cells are treated with LNP formulated mRNAs at decreasing doses and cell viability is measured by Cell Titer Glo protocol.

### Example 4: Further Induction of Cell Death by Translatable Nucleic Acid Constructs

The present example further demonstrates the effective killing of mammalian cells by translatable nucleic acids. Specifically, the present example confirms that introduction of an in vitro transcribed mRNA encoding a constitutively active suicide protein into mammalian cell results in immunogenic cell death.

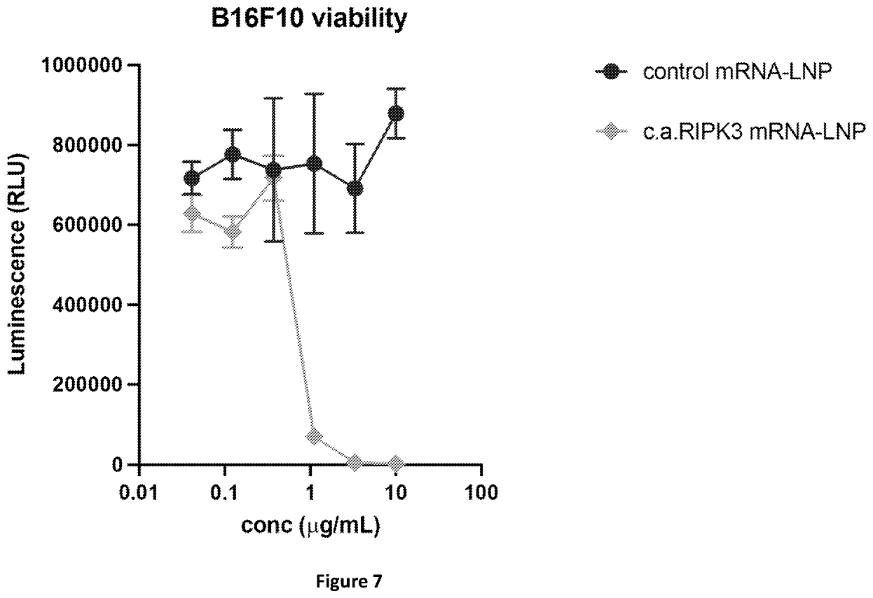

To assess the ability of a constitutively active suicide protein expressed from an mRNA to induce cell death B16F10 tumor cells were incubated for 48 hours with serially diluted caRIPK3 mRNA-LNP or control mRNA-LNP. Cell viability plotted as RLU (Relative Luminescence Units) was assessed by quantitation of ATP present as indicator of metabolically active cells (viable cells) using the Cell-Titer glow luminescent cell viability assay. FIG. 7 confirms that a constitutively active RIPK3 is capable of inducing cancer cell death.

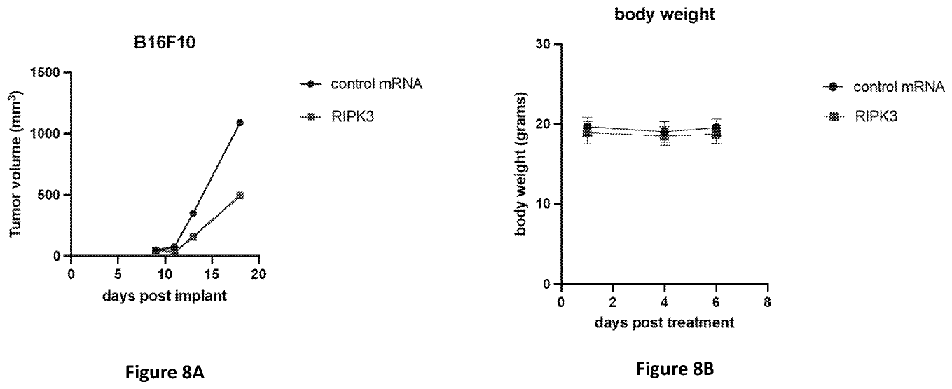

Further, female B16 albino mice were inoculated subcutaneously with B16F10 tumor cells. Intra-tumoral treatment with 13.3 mg of mRNA started when tumors reached approximately 50-60 mm³ and was repeated every 2-3 days for a total of 3 doses. Data illustrated shows median of tumor growth (n=5) over the course of 9 days from treatment. Treatment with caRIPK3 mRNA considerably reduced tumor volume over the treatment period (see FIG. 8)further confirming that a constitutively active RIPK3 is capable of inducing cancer cell death.

### Example 5: Sequences

The present example provides sequences of constructs used herein.

U27-Gasdermin
(SEQ ID NO. 1)
GGAAATAAGAGAGAAAAGAAGAGTAAGAAGAAATATAAGA
GCCACCatgAAGTGAGGCTCTCCTCCCGCCCCGCCCCTCC
CACGCCTCACCAGCCCCCCGCGCGCCCACCCTCCGGCGGG
TGACAGCTCCGGGATCAGCAACCCTTCCTGCTGCTGCTAC
TGCTGCTGCTGCTGCCGCCGCCGCCGCCGCCGCTGCCCTT
GGGTCCCCCCGAGTCTCCGGGACTGCCCTCTCGACTGTCA
GTGGGGCAGCCTCTCCGACTCTGCACCCGCCTCGACCTCC
CCACCCGCTCCCACACCCCTGTGCCCTCATGTGGAGCCTA
AGAGAACAGAACAGGCCGTGAAGCCAGCAGAGAAAggaag
cggagctactaacttcagcctgctgaagcaggctggagac
gtggaggagaaccctggaccttactcgagcagACGCGTag
atctgGGCTCCGGCGAGGGCAGGGGAAGTCTTCTAACATG
CGGGGACGTGGAGGAAAATCCCGGCCCAGGCAGCGCCTTC
GAGCGCGTCGTCCGCCGCGTCGTCCAGGAGCTCGACCACG
GCGGCGAGTTCATCCCCGTCACCAGCCTCCAGAGCAGCAC
CGGCTTCCAGCCCTACTGCCTCGTCGTCCGCAAGCCCAGC
AGCAGCTGGTTCTGGAAGCCCCGCTACAAGTGCGTGAACC
TGAGCATCAAGGACATCCTCGAGCCGGACGCCGCCGAGCC
CGACGTCCAGCGCGGCCGCAGCTTCCACTTCTACGACGCC
ATGGACGGCCAGATCCAGGGCAGCGTCGAGCTGGCCGCCC
CCGGCCAGGCCAAGATCGCCGGCGGCGCCGCCGTCAGCGA
CAGCAGCAGCACCAGCATGAACGTCTACAGCCTCAGCGTC
GACCCCAACACCTGGCAGACCCTCCTCCACGAGCGCCACC
TCCGCCAGCCCGAGCACAAGGTCCTCCAGCAGCTCCGCAG
CCGGGGCGACAACGTCTACGTCGTCACCGAGGTCCTCCAG
ACCCAGAAGGAGGTCGAGGTCACCCGGACCCACAAGCGCG
AGGGCAGCGGCCGCTTCAGCCTCCCCGGCGCCACCTGCCT
CCAGGGCGAGGGCCAGGGCCACCTCAGCCAGAAGAAGACC
GTCACCATCCCCAGCGGCAGCACCCTCGCCTTCCGCGTCG
CCCAGCTCGTCATCGACAGCGACCTCGACGTCCTCCTCTT
CCCCGACAAGAAGCAGCGCACCTTCCAGCCCCCCGCCACC
GGCCACAAGCGCAGCACCAGCGAGGGCGCCTGGCCCCAGC
TCCCCAGCGGCCTCAGCATGATGCGCTGCCTCCACAACTT
CCTCACCGACTGATAATTAAACCGGTTTAAGCTGCCTTCT
GCGGGGCTTGCCTTCTGGCCATGCCCTTCTTCTCTCCCTT
GCACCTGTACCTCTTGGTCTTTGAATAAAGCCTGAGTAGG
AAGAAGCCTGCATGCCTGGTTCTCTGCGTCTGCGAATTCG
ATATCCAGCGGCCGCGCTAGCGCTG
caMLKL
(SEQ ID NO. 2)
ggTCAAGCTTTTGGACCCTCGTACAGAAGCTAATACGACT
CACTATAGGGAAATAAGAGAGAAAAGAAGAGTAAGAAGAA
ATATAAGAGCCACCATGGAAAATTTGAAGCATATTATCAC
CCTTGGCCAGGTCATCCACAAACGGTGTGAAGAGATGAAA
TACTGCAAGAAACAGTGCCGGCGCCTGGGCCACCGCGTCC
TCGGCCTGATCAAGCCTCTGGAGATGCTCCAGGACCAAGG
AAAGAGGAGCGTGCCCTCTGAGAAGTTAACCACAGCCATG
AACCGCTTCAAGGCTGCCCTGGAGGAGGCTAATGGGGAGA
TAGAAAAGTTCAGCAATAGATCCAATATCTGCAGGTTTCT
AACAGCAAGCCAGGACAAAATACTCTTCAAGGACGTGAAC
AGGAAGCTGAGTGATGTCTGGAAGGAGCTCTCGCTGTTAC
TTCAGGTTGAGCAACGCATGCCTGTTTCACCCATAAGCCA
AGGAGCGTCCTGGGCACAGGAAGATCAGCAGGATGCAGAC
GAAGACAGGCGAGCTTTCCAGATGCTAAGAAGAGATAATG
AAAAAATAGAAGCTTCACTGAGACGATTAGAAATCAACAT
GAAAGAAATCAAGGAAACTTTGAGGCAGTATTTACCACCA
AAATGCATGCAGGAGATCCCGCAAGAGCAAATCAAGGAGA
TCAAGAAGGAGCAGCTTTCAGGATCCCCGTGGATTCTGCT
AAGGGAAAATGAAGTCAGCACACTTTATAAAGGAGAATAC
CACAGAGCTCCAGTGGCCATAATGGTATTCAAAAAACTCC
AGGCTGGCAGCATTGCAATAGTGAGGCAGACTTTCAATAA
GGAGATCAAAACCATGAAGAAATTCGAATCTCCCAACATC
CTGCGTATATTTGGGATTTGCATTGATGAAACAGTGACTC
CGCCTCAATTCTCCATTGTCATGGAGTACTGTGAACTCGG
GACCCTGAGGGAGCTGTTGGATAGGGAAAAAGACCTCACA
CTTGGCAAGCGCATGGTCCTAGTCCTGGGGGCAGCCCGAG
GCCTATACCGGCTACACCATTCAGAAGCACCTGAACTCCA
CGGAAAAATCAGAAGCTCAAACTTCCTGGTAACTCAAGGC
TACCAAGTGAAGCTTGCAGGATTTGAGTTGAGGAAAACAG
CCACTTCCATGAGTTTGGGAACTACGAGAGAAAAGACAGA
CAGAGTCAAATCTACAGCATATCTCTCACCTCAGGAACTG
GAAGATGTATTTTATCAATATGATGTAAAGTCTGAAATAT
ACAGCTTTGGAATCGTCCTCTGGGAAATCGCCACTGGAGA
TATCCCGTTTCAAGGCTGTAATTCTGAGAAGATCCGCAAG
CTGGTGGCTGTGAAGCGGCAGCAGGAGCCACTGGGTGAAG
ACTGCCCTTCAGAGCTGCGGGAGATCATTGATGAGTGCCG
GGCCCATGATCCCTCTGTGCGGCCCTCTGTGGATGAAATC
TTAAAGAAACTCTCCACCTTTTCTAAGTGATTAATTAAGC
TGCCTTCTGCGGGGCTTGCCTTCTGGCCATGCCCTTCTTC
TCTCCCTTGCACCTGTACCTCTTGGTCTTTGAATAAAGCC
TGAGTAGGAAGAAGCCTGCATGCCTGGTTCTCTGCGTCTG
CGAATTCGATATCCAGCGGCCGC
caGasdermin
(SEQ ID NO. 3)
GGAAATAAGAGAGAAAAGAAGAGTAAGAAGAAATATAAGA
GCCACCATGGGGTCGGCCTTTGAGCGGGTAGTCCGGAGAG
TGGTCCAGGAGCTGGACCATGGTGGGGAGTTCATCCCTGT
GACCAGCCTGCAGAGCTCCACTGGCTTCCAGCCCTACTGC
CTGGTGGTTAGGAAGCCCTCAAGCTCATGGTTCTGGAAAC
CCCGTTATAAGTGTGTCAACCTGTCTATCAAGGACATCCT
GGAGCCGGATGCCGCGGAACCAGACGTGCAGCGTGGCAGG
AGCTTCCACTTCTACGATGCCATGGATGGGCAGATACAGG
GCAGCGTGGAGCTGGCAGCCCCAGGACAGGCAAAGATCGC
AGGCGGGGCCGCGGTGTCTGACAGCTCCAGCACCTCAATG
AATGTGTACTCGCTGAGTGTGGACCCTAACACCTGGCAGA
CTCTGCTCCATGAGAGGCACCTGCGGCAGCCAGAACACAA
AGTCCTGCAGCAGCTGCGCAGCCGCGGGGACAACGTGTAC
GTGGTGACTGAGGTGCTGCAGACACAGAAGGAGGTGGAAG
TCACGCGCACCCACAAGCGGGAGGGCTCGGGCCGGTTTTC
CCTGCCCGGAGCCACGTGCTTGCAGGGTGAGGGCCAGGGC
CATCTGAGCCAGAAGAAGACGGTCACCATCCCCTCAGGCA
GCACCCTCGCATTCCGGGTGGCCCAGCTGGTTATTGACTC
TGACTTGGACGTCCTTCTCTTCCCGGATAAGAAGCAGAGG
ACCTTCCAGCCACCCGCGACAGGCCACAAGCGTTCCACGA
GCGAAGGCGCCTGGCCACAGCTGCCCTCTGGCCTCTCCAT
GATGAGGTGCCTCCACAACTTCCTGACAGATTGATTAATT
AAGCTGCCTTCTGCGGGGCTTGCCTTCTGGCCATGCCCTT
CTTCTCTCCCTTGCACCTGTACCTCTTGGTCTTTGAATAA
AGCCTGAGTAGGAAGAAGCCTGCATGCCTGGTTCTCTGCG
TCTGCGAATTCGATATCCAGCGGCCGC
Fluc-T2A-MLKL (S345D, S347D)
(SEQ ID NO. 4)
GAAATAAGAGAGAAAAGAAGAGTAAGAAGAAATATAAGAG
CCACCatggaagacgccaaaaacataaagaaaggcccggc
gccattctatccgctggaagatggaaccgctggaGagcaa
ctgcataaggctatgaagagatacgccctggttcctggaa
caattgcttttacagatgcacatatcgaggtggacatcac
ttacgctgagtacttcgaaatgtccgttcggttggcagaa
gctatgaaacgatatgggctgaatacaaatcacagaatcg
tcgtatgcagtgaaaactctcttcaattctttatgccggt
gttgggcgcgttatttatcggagttgcagttgcgcccgcg
aacgacatttataatgaacgtgaattgctcaacagtatgg
gcatttcgcagcctaccgtggtgttcgtttccaaaaaggg
gttgcaaaaaattttgaacgtgcaaaaaaagctcccaatc
atccaaaaaattattatcatggattctaaaacggattacc
agggatttcagtcgatgtacacgttcgtcacatctcatct
acctcccggttttaatgaatacgattttgtgccagagtcc
ttcgatagggacaagacaattgcactgatcatgaactcct
ctggatctactggtctgcctaaaggtgtcgctctgcctca
tagaactgcctgcgtgagattctcgcatgccagagatcct
atttttggcaatcaaatcattccggatactgcgattttaa
gtgttgttccattccatcacggttttggaatgtttactac
actcggatatttgatatgtggatttcgagtcgtcttaatg
tatagatttgaagaagagctgtttctgaggagccttcagg
attacaagattcaaagtgcgctgctggtgccaaccctatt
ctccttcttcgccaaaagcactctgattgacaaatacgat
ttatctaatttacacgaaattgcttctggtggcgctcccc
tctctaaggaagtcggggaagcggttgccaagaggttcca
tctgccaggtatcaggcaaggatatgggctcactgagact
acatcagctattctgattacacccgagggggatgataaac
cgggcgcggtcggtaaagttgttccattttttgaagcgaa
ggttgtggatctggataccgggaaaacgctgggcgttaat
caaagaggcgaactgtgtgtgagaggtcctatgattatgt
ccggttatgtaaacaatccggaagcgaccaacgccttgat
tgacaaggatggatggctacattctggagacatagcttac
tgggacgaagacgaacacttcttcatcgttgaccgcctga
agtctctgattaagtacaaaggctatcaggtggctcccgc
tgaattggaatccatcttgctccaacaccccaacatcttc
gacgcaggtgtcgcaggtcttcccgacgatgacgccggtg
aacttcccgccgccgttgttgttttggagcacggaaagac
gatgacggaaaaagagatcgtggattacgtcgccagtcaa
gtaacaaccgcgaaaaagttgcgcggaggagttgtgtttg
tggacgaagtaccgaaaggtcttaccggaaaactcgacgc
aagaaaaatcagagagatcctcataaaggccaagaagggc
ggaaagatcgccgtgGGCTCCGGCGAGGGCAGGGGAAGTC
TTCTAACATGCGGGGACGTGGAGGAAAATCCCGGCCCAAT
GGATAAATTGGGACAGATCATCAAGTTAGGCCAGCTCATC
TATGAACAGTGTGAAAAGATGAAATACTGCCGGAAACAAT
GCCAGCGTCTAGGAAACCGTGTGCACGGCCTGCTACAGCC
TCTCCAGAGACTCCAGGCCCAAGGAAAGAAGAACCTGCCC
GATGACATTACTGCTGCCCTGGGCCGTTTTGATGAAGTCC
TGAAGGAGGCTAACCAGCAGATAGAAAAGTTCAGCAAGAA
GTCCCATATTTGGAAGTTTGTGAGTGTGGGCAATGATAAG
ATCCTCTTCCATGAAGTGAATGAGAAGCTGAGAGACGTCT
GGGAGGAGCTGTTGCTGCTGCTTCAGGTTTATCATTGGAA
TACCGTTTCAGATGTCAGCCAGCCAGCATCCTGGCAGCAG
GAAGATCGACAGGATGCAGAGGAAGACGGAAATGAAAATA
TGAAAGTTATCCTGATGCAGTTGCAAATTAGCGTGGAAGA
AATCAACAAAACCCTGAAGCAATGCTCACTAAAACCCACA
CAGGAGATCCCACAAGATCTCCAAATCAAGGAGATTCCAA
AGGAACATCTTGGACCTCCGTGGACCAAACTGAAGACAAG
TAAAATGAGCACCATTTATAGAGGAGAGTATCACAGATCT
CCAGTTACCATCAAAGTATTCAACAACCCCCAGGCCGAAA
GTGTTGGAATAGTGAGGTTCACTTTCAATGACGAGATCAA
AACCATGAAGAAATTCGATTCTCCCAACATCTTGCGTATA
TTTGGGATTTGCATTGATCAAACAGTGAAGCCCCCTGAGT
TCTCCATTGTCATGGAGTACTGTGAACTTGGAACCCTGAG
GGAACTGCTGGATAGAGAAAAAGACCTCACAATGAGTGTG
CGCAGCCTCCTAGTCCTGAGGGCAGCCAGAGGCTTATACA
GGCTACACCATTCGGAAACACTCCACAGAAACATCAGCAG
CTCCAGTTTCCTCGTAGCCGGAGGCTACCAAGTAAAGCTT
GCAGGATTTGAGTTAAGCAAAACACAGAATGATATCGACC
GGACAGCAAAGAGCACTAAAGCAGAGAGATCCAGTTCAAC
GATATATGTCTCCCCTGAGAGACTGAAAAATCCATTTTGC
CTTTATGACATAAAAGCTGAAATATATAGCTTTGGAATTG
TACTCTGGGAAATTGCCACTGGAAAGATCCCATTTGAAGG
CTGTGATTCTAAGAAGATCCGGGAGCTGGTGGCTGAGGAC
AAGAAGCAGGAACCAGTGGGTCAGGATTGCCCTGAGTTGT
TGCGGGAAATCATTAATGAGTGTCGTGCCCATGAGCCCTC
CCAACGGCCCTCTGTGGACGGTAGGAGTCTTTCTGGCAGA
GAACGAATCTTGGAGAGACTGTCTGCGGTTGAAGAATCCA
CGGACAAGAAGGTGTAAGCTGCCTTCTGCGGGGCTTGCCT
TCTGGCCATGCCCTTCTTCTCTCCCTTGCACCTGTACCTC
TTGGTCTTTGAATAAAGCCTGAGTAGGAAG
caRIPK3 mouse
(SEQ ID NO. 5)
AGGTCTTCTGGTCCCCACAGACTCAGAGAGAACCCGCCAC
CATGAGCAGCGTGAAGCTGTGGCCTACCGGCGCCAGCGCC
GTGCCCCTGGTGAGCCGGGAGGAGTTAAAGAAGCTGGAGT
TCGTGGGCAAGGGCGGCTTCGGCGTGGTGTTCCGGGCCCA
CCACCGGACTTGGAACCACGATGTGGCCGTGAAGATCGTG
AATAGCAAGAAGATCAGCTGGGAGGTGAAGGCCATGGTGA
ACCTGCGGAACGAGAACGTGCTGCTGCTGCTGGGCGTGAC
CGAGGACCTGCAGTGGGACTTCGTGAGCGGCCAGGCCCTG
GTGACCCGGTTCATGGAGAATGGCAGCCTGGCCGGCCTGC
TGCAGCCCGAGTGCCCTCGGCCCTGGCCCCTCCTGTGCCG
GCTGCTGCAGGAGGTGGTGCTGGGCATGTGCTACCTGCAC
AGCCTGAACCCACCCCTGCTGCACAGGGACCTGAAGCCCA
GCAACATCCTGCTGGACCCTGAGCTGCACGCCAAGCTGGC
CGATTTCGGCCTGAGCACCTTCCAGGGCGGCAGCCAGAGC
GGCAGCGGCAGCGGAAGCGGCTCACGGGACAGCGGCGGCA
CCCTGGCCTACCTGGACCCCGAGCTGCTGTTCGACGTGAA
CCTGAAGGCCAGCAAAGCCAGCGACGTGTACAGCTTCGGC
ATCCTGGTGTGGGCCGTGCTGGCAGGCCGGGAGGCTGAGC
TGGTGGACAAGACCAGCCTGATCCGGGAGACCGTGTGCGA
CCGGCAGAGCCGGCCCCCCCTGACCGAGCTGCCCCCCGGC
AGCCCCGAGACCCCCGGCCTGGAGAAGCTCAAGGAGCTGA
TGATCCACTGCTGGGGCTCACAGTCCGAGAATCGTCCAAG
CTTCCAGGACTGCGAGCCCAAGACCAACGAGGTGTACAAC
CTGGTGAAGGACAAGGTGGACGCCGCCGTGAGCGAGGTGA
AGCACTACCTGAGCCAGCACCGGAGCAGCGGCCGGAACCT
GAGCGCCCGGGAGCCCAGCCAGCGGGGCACCGAGATGGAC
TGCCCCCGGGAGACCATGGTGAGCAAGATGCTGGACCGGC
TGCACCTGGAGGAGCCCAGCGGACCTGTGCCCGGCAAGTG
CCCCGAGCGGCAGGCCCAGGACACCAGCGTGGGCCCCGCC
ACCCCCGCCCGGACCAGCAGCGACCCCGTGGCCGGCACCC
CTCAGATCCCCCACACCCTGCCCTTCCGGGGCACCACCCC
CGGCCCCGTGTTCACCGAGACCCCAGGCCCCCACCCCCAG
AGGAATCAGGGCGACGGCCGGCACGGCACCCCATGGTACC
CTTGGACCCCCCCCAACCCCATGACCGGCCCCCCCGCACT
GGTGTTTAATAACTGTAGCGAGGTGCAGATCGGCAACTAC
AACAGCCTGGTCGCCCCCCCCCGGACCACCGCCAGCAGCA
GCGCCAAGTACGACCAGGCCCAGTTCGGCCGGGGCCGGGG
CTGGCAGCCCTTCCACAAGGGCACCAAGTACGAGATCGAA
CGGATGCTGAAGGAGCTGAAGGACCTGCTGGAAGAGCTGA
AGCGGATGCTGGAGGAGCTTAAACGGAGCCTGAAGGAACT
GAAGAAGAACCCCAGCGAGGACGCCCTGGTGGAGAATAAC
CGGCTGATCGTGAAGGTGCTGGAGGTGATCGTGGAGGTGC
TGCGGGCCATCATCGAGCTGGCCGAGCTGATCATCCGGAG
CGACTGATTAAACCGGTCTGCAGCTTTTGCCTTGGTTCTT
CCTAGTGCCTACAATGGGAAAACTTCACTCCAAAGAGAAA
CCTATTAAGTCATCATCTCCAAACTAAACCCTCACAAGTA
ACAGTTGAAGAAAAAATGGCAAGAGATCATATCCTCAGAC
CAGGTGGAATTACTTAAATTTTAAAGCCTGAAAATTCCAA
TTTGGGGGTGGGAGGTGGAAAAGGAACCCAATTTTCTTAT
GAACAGATATTTTTAACTTAATGGCACAAAGTCTTAGAAT
ATTATTATGTGCCCCGTGTTCCCTGTTCTTCGTTGCTGAA
TTCGATATCCAGCGGCCGCGCTAGCGCTG
caRIPK3 human
(SEQ ID NO. 6)
AGGTCTTCTGGTCCCCACAGACTCAGAGAGAACCCGCCAC
CATGAGCTGCGTGAAGCTGTGGCCCAGCGGCGCCCCCGCC
CCACTGGTGAGCATCGAGGAGCTGGAGAACCAGGAGCTGG
TGGGCAAGGGCGGCTTCGGCACCGTGTTCCGGGCCCAGCA
CCGGAAGTGGGGCTACGACGTGGCCGTGAAGATCGTGAAC
AGCAAGGCAATCAGCCGGGAGGTGAAGGCCATGGCCAGCC
TGGACAACGAGTTCGTGCTGCGGCTGGAGGGCGTGATCGA
GAAGGTGAACTGGGACCAGGACCCAAAGCCTGCCCTGGTG
ACCAAGTTCATGGAGAACGGCAGCCTGAGCGGACTGCTGC
AGAGCCAGTGCCCCCGGCCCTGGCCACTGCTGTGCCGGCT
GCTGAAGGAGGTGGTGCTGGGCATGTTCTACCTGCACGAT
CAGAACCCCGTGCTGCTGCACCGTGACCTGAAGCCCAGCA
ACGTGCTGCTGGACCCCGAGCTGCACGTGAAGCTGGCTGA
CTTCGGCCTGAGCACCTTCCAGGGCGGCAGCCAGAGCGGC
ACCGGCAGCGGCGAGCCCGGCGGCACCCTGGGCTACCTGG
CCCCCGAGCTGTTCGTGAATGTGAACCGGAAGGCCAGCAC
CGCCAGCGACGTGTACAGCTTCGGCATCCTGATGTGGGCC
GTGCTGGCCGGCCGGGAGGTCGAGCTGCCCACCGAGCCCA
GCCTGGTGTACGAGGCTGTGTGCAACCGGCAGAACCGGCC
CAGCCTGGCAGAGCTGCCCCAGGCCGGCCCCGAGACCCCC
GGCCTGGAGGGCCTTAAGGAGCTGATGCAGCTGTGCTGGA
GCAGCGAGCCTAAGGACCGGCCCAGCTTCCAGGAGTGCCT
GCCCAAGACCGACGAGGTGTTCCAGATGGTCGAGAATAAT
ATGAACGCAGCTGTGAGCACCGTGAAGGACTTCCTGAGCC
AGCTGCGGAGCAGCAACCGGCGGTTCAGCATCCCCGAGAG
CGGCCAGGGAGGCACCGAGATGGATGGCTTCCGGCGGACC
ATCGAGAACCAGCACAGCAGGAATGACGTGATGGTGAGCG
AGTGGCTGAACAAGCTGAACCTGGAGGAGCCCCCCAGCAG
CGTGCCAAAGAAGTGCCCCAGCCTGACCAAGCGGAGCCGG
GCCCAGGAGGAGCAGGTGCCCCAGGCCTGGACCGCCGGCA
CCAGCAGCGACAGCATGGCCCAGCCCCCCCAGACCCCAGA
GACCTCAACCTTCCGGAACCAGATGCCCAGCCCCACCAGC
ACCGGCACCCCAAGCCCCGGCCCCCGGGGCAACCAGGGCG
CCGAGAGGCAGGGCATGAACTGGAGCTGCCGGACCCCCGA
GCCCAACCCCGTGACCGGCCGGCCCCTGGTGAACATCTAC
AACTGCAGCGGCGTGCAGGTGGGAGACAACAACTACCTGA
CCATGCAGCAGACCACCGCTCTGCCCACCTGGGGCCTGGC
CCCAAGCGGCAAGGGCCGGGGCCTGCAGCACCCCCCCCCC
GTGGGCAGCCAGGAGGGCCCCAAGGACCCAGAGGCCTGGA
GCCGGCCCCAGGGCTGGTACAACCACAGCGGCAAGGGGAC
CAAGTACGAGATCGAGCGGATGCTGAAGGAACTGAAGGAC
CTGCTGGAGGAGCTGAAACGGATGCTCGAGGAGCTGAAGC
GGAGCCTGAAGGAGCTGAAGAAGAACCCAAGCGAGGACGC
CCTGGTGGAGAACAACCGGCTGATCGTGAAGGTGCTGGAG
GTGATCGTGGAGGTGCTGCGGGCCATCATCGAGCTGGCCG
AGCTGATAATCCGGAGCGACTGATTAAACCGGTCTGCAGC
TTTTGCCTTGGTTCTTCCTAGTGCCTACAATGGGAAAACT
TCACTCCAAAGAGAAACCTATTAAGTCATCATCTCCAAAC
TAAACCCTCACAAGTAACAGTTGAAGAAAAAATGGCAAGA
GATCATATCCTCAGACCAGGTGGAATTACTTAAATTTTAA
AGCCTGAAAATTCCAATTTGGGGGTGGGAGGTGGAAAAGG
AACCCAATTTTCTTATGAACAGATATTTTTAACTTAATGG
CACAAAGTCTTAGAATATTATTATGTGCCCCGTGTTCCCT
GTTCTTCGTTGCTGAATTCGATATCCAGCGGCCGCGCTAG
CGCTG
ca-human-Gasdermin
(SEQ ID NO. 7)
AGGUCUUCUGGUCCCCACAGACUCAGAGAGAACCCGCCAC
CaugGGCAGCGCCUUCGAGCGCGUCGUCCGCCGCGUCGUC
CAGGAGCUCGACCACGGCGGCGAGUUCAUCCCCGUCACCA
GCCUCCAGAGCAGCACCGGCUUCCAGCCCUACUGCCUCGU
CGUCCGCAAGCCCAGCAGCAGCUGGUUCUGGAAGCCCCGC
UACAAGUGCGUGAACCUGAGCAUCAAGGACAUCCUCGAGC
CGGACGCCGCCGAGCCCGACGUCCAGCGCGGCCGCAGCUU
CCACUUCUACGACGCCAUGGACGGCCAGAUCCAGGGCAGC
GUCGAGCUGGCCGCCCCCGGCCAGGCCAAGAUCGCCGGCG
GCGCCGCCGUCAGCGACAGCAGCAGCACCAGCAUGAACGU
CUACAGCCUCAGCGUCGACCCCAACACCUGGCAGACCCUC
CUCCACGAGCGCCACCUCCGCCAGCCCGAGCACAAGGUCC
UCCAGCAGCUCCGCAGCCGGGGCGACAACGUCUACGUCGU
CACCGAGGUCCUCCAGACCCAGAAGGAGGUCGAGGUCACC
CGGACCCACAAGCGCGAGGGCAGCGGCCGCUUCAGCCUCC
CCGGCGCCACCUGCCUCCAGGGCGAGGGCCAGGGCCACCU
CAGCCAGAAGAAGACCGUCACCAUCCCCAGCGGCAGCACC
CUCGCCUUCCGCGUCGCCCAGCUCGUCAUCGACAGCGACC
UCGACGUCCUCCUCUUCCCCGACAAGAAGCAGCGCACCUU
CCAGCCCCCCGCCACCGGCCACAAGCGCAGCACCAGCGAG
GGCGCCUGGCCCCAGCUCCCCAGCGGCCUCAGCAUGAUGC
GCUGCCUCCACAACUUCCUCACCGACUGAUUAAACCGGUC
UGCAGCUUUUGCCUUGGUUCUUCCUAGUGCCUACAAUGGG
AAAACUUCACUCCAAAGAGAAACCUAUUAAGUCAUCAUCU
CCAAACUAAACCCUCACAAGUAACAGUUGAAGAAAAAAUG
GCAAGAGAUCAUAUCCUCAGACCAGGUGGAAUUACUUAAA
UUUUAAAGCCUGAAAAUUCCAAUUUGGGGGUGGGAGGUGG
AAAAGGAACCCAAUUUUCUUAUGAACAGAUAUUUUUAACU
UAAUGGCACAAAGUCUUAGAAUAUUAUUAUGUGCCCCGUG
UUCCCUGUUCUUCGUUGCUGAAUUCGAUAUCCAGCGGCCG
CGCUAGCGCUG
mouse-Gasdermin
(SEQ ID NO. 8)
AGGUCUUCUGGUCCCCACAGACUCAGAGAGAACCCGCCAC
CaugCCAAGCGCCUUCGAGAAGGUGGUGAAGAACGUGAUC
AAGGAGGUCUCCGGCAGCCGGGGCGACCUGAUCCCCGUGG
ACAGCCUGCGGAACAGCACCAGCUUCCGGCCCUACUGCCU
GCUGAACCGGAAGUUCAGCAGCAGCCGGUUCUGGAAGCCC
CGGUACAGCUGCGUGAACCUGAGCAUCAAGGACAUCCUGG
AGCCCAGCGCCCCCGAGCCCGAGCCUGAGUGUUUCGGCAG
CUUCAAGGUGAGCGACGUGGUGGACGGCAACAUCCAGGGC
CGGGUGAUGCUGAGCGGCAUGGGCGAGGGAAAGAUCAGCG
GGGGCGCCGCCGUGAGCGACAGCAGCAGCGCCAGCAUGAA
CGUGUGCAUCCUGCGGGUGACCCAGAAGACCUGGGAGACC
AUGCAGCACGAGCGGCACCUGCAGCAGCCUGAGAAUAAGA
UCCUGCAGCAGCUGCGGAGCCGGGGCGAUGACCUGUUCGU
GGUGACCGAAGUGCUGCAGACCAAGGAGGAGGUGCAGAUC
ACCGAGGUGCACAGCCAGGAGGGCAGCGGCCAGUUCACCC
UGCCCGGCGCCCUGUGCCUGAAGGGCGAGGGCAAGGGACA
CCAGAGCCGGAAGAAGAUGGUGACCAUCCCCGCCGGCAGC
AUCCUGGCCUUCCGGGUGGCCCAGCUGCUGAUCGGCAGCA
AGUGGGACAUUCUGCUGGUGAGCGACGAGAAGCAGCGGAC
CUUCGAGCCCAGCAGCGGCGACCGGAAGGCCGUGGGCCAG
CGGCACCACGGCCUGAACGUGCUGGCCGCCCUGUGUAGCA
UCGGCAAGCAGCUAAGCCUGCUGAGCGACUGAUUAAACCG
GUCUGCAGCUUUUGCCUUGGUUCUUCCUAGUGCCUACAAU
GGGAAAACUUCACUCCAAAGAGAAACCUAUUAAGUCAUCA
UCUCCAAACUAAACCCUCACAAGUAACAGUUGAAGAAAAA
AUGGCAAGAGAUCAUAUCCUCAGACCAGGUGGAAUUACUU
AAAUUUUAAAGCCUGAAAAUUCCAAUUUGGGGGUGGGAGG
UGGAAAAGGAACCCAAUUUUCUUAUGAACAGAUAUUUUUA
ACUUAAUGGCACAAAGUCUUAGAAUAUUAUUAUGUGCCCC
GUGUUCCCUGUUCUUCGUUGCUGAAUUCGAUAUCCAGCGG
CCGCGCUAGCGCUG
U-27-ca-human Gasdermin
(SEQ ID NO. 9)
AGGUCUUCUGGUCCCCACAGACUCAGAGAGAACCCGCCAC
CaugAAGUGAGGCUCUCCUCCCGCCCCGCCCCUCCCACGC
CUCACCAGCCCCCCGCGCGCCCACCCUCCGGCGGGUGACA
GCUCCGGGAUCAGCAACCCUUCCUGCUGCUGCUACUGCUG
CUGCUGCUGCCGCCGCCGCCGCCGCCGCUGCCCUUGGGUC
CCCCCGAGUCUCCGGGACUGCCCUCUCGACUGUCAGUGGG
GCAGCCUCUCCGACUCUGCACCCGCCUCGACCUCCCCACC
CGCUCCCACACCCCUGUGCCCUCAUGUGGAGCCUAAGAGA
ACAGAACAGGCCGUGAAGCCAGCAGAGAAAggaagcggag
cuacuaacuucagccugcugaagcaggcuggagacgugga
ggagaacccuggaccuuacucgagcagACGCGUagaucug
GGCUCCGGCGAGGGCAGGGGAAGUCUUCUAACAUGCGGGG
ACGUGGAGGAAAAUCCCGGCCCAGGCAGCGCCUUCGAGCG
CGUCGUCCGCCGCGUCGUCCAGGAGCUCGACCACGGCGGC
GAGUUCAUCCCCGUCACCAGCCUCCAGAGCAGCACCGGCU
UCCAGCCCUACUGCCUCGUCGUCCGCAAGCCCAGCAGCAG
CUGGUUCUGGAAGCCCCGCUACAAGUGCGUGAACCUGAGC
AUCAAGGACAUCCUCGAGCCGGACGCCGCCGAGCCCGACG
UCCAGCGCGGCCGCAGCUUCCACUUCUACGACGCCAUGGA
CGGCCAGAUCCAGGGCAGCGUCGAGCUGGCCGCCCCCGGC
CAGGCCAAGAUCGCCGGCGGCGCCGCCGUCAGCGACAGCA
GCAGCACCAGCAUGAACGUCUACAGCCUCAGCGUCGACCC
CAACACCUGGCAGACCCUCCUCCACGAGCGCCACCUCCGC
CAGCCCGAGCACAAGGUCCUCCAGCAGCUCCGCAGCCGGG
GCGACAACGUCUACGUCGUCACCGAGGUCCUCCAGACCCA
GAAGGAGGUCGAGGUCACCCGGACCCACAAGCGCGAGGGC
AGCGGCCGCUUCAGCCUCCCCGGCGCCACCUGCCUCCAGG
GCGAGGGCCAGGGCCACCUCAGCCAGAAGAAGACCGUCAC
CAUCCCCAGCGGCAGCACCCUCGCCUUCCGCGUCGCCCAG
CUCGUCAUCGACAGCGACCUCGACGUCCUCCUCUUCCCCG
ACAAGAAGCAGCGCACCUUCCAGCCCCCCGCCACCGGCCA
CAAGCGCAGCACCAGCGAGGGCGCCUGGCCCCAGCUCCCC
AGCGGCCUCAGCAUGAUGCGCUGCCUCCACAACUUCCUCA
CCGACUGAUUAAACCGGUCUGCAGCUUUUGCCUUGGUUCU
UCCUAGUGCCUACAAUGGGAAAACUUCACUCCAAAGAGAA
ACCUAUUAAGUCAUCAUCUCCAAACUAAACCCUCACAAGU
AACAGUUGAAGAAAAAAUGGCAAGAGAUCAUAUCCUCAGA
CCAGGUGGAAUUACUUAAAUUUUAAAGCCUGAAAAUUCCA
AUUUGGGGGUGGGAGGUGGAAAAGGAACCCAAUUUUCUUA
UGAACAGAUAUUUUUAACUUAAUGGCACAAAGUCUUAGAA
UAUUAUUAUGUGCCCCGUGUUCCCUGUUCUUCGUUGCUGA
AUUCGAUAUCCAGCGGCCGCGCUAGCGCUG
U-27 mouse Gasdermin
(SEQ ID NO. 10)
AGGUCUUCUGGUCCCCACAGACUCAGAGAGAACCCGCCAC
CaugAAGUGAGGCUCUCCUCCCGCCCCGCCCCUCCCACGC
CUCACCAGCCCCCCGCGCGCCCACCCUCCGGCGGGUGACA
GCUCCGGGAUCAGCAACCCUUCCUGCUGCUGCUACUGCUG
CUGCUGCUGCCGCCGCCGCCGCCGCCGCUGCCCUUGGGUC
CCCCCGAGUCUCCGGGACUGCCCUCUCGACUGUCAGUGGG
GCAGCCUCUCCGACUCUGCACCCGCCUCGACCUCCCCACC
CGCUCCCACACCCCUGUGCCCUCAUGUGGAGCCUAAGAGA
ACAGAACAGGCCGUGAAGCCAGCAGAGAAAggaagcggag
cuacuaacuucagccugcugaagcaggcuggagacgugga
ggagaacccuggaccuuacucgagcagACGCGUagaucug
GGCUCCGGCGAGGGCAGGGGAAGUCUUCUAACAUGCGGGG
ACGUGGAGGAAAAUCCCGGCCCACCAAGCGCCUUCGAGAA
GGUGGUGAAGAACGUGAUCAAGGAGGUCUCCGGCAGCCGG
GGCGACCUGAUCCCCGUGGACAGCCUGCGGAACAGCACCA
GCUUCCGGCCCUACUGCCUGCUGAACCGGAAGUUCAGCAG
CAGCCGGUUCUGGAAGCCCCGGUACAGCUGCGUGAACCUG
AGCAUCAAGGACAUCCUGGAGCCCAGCGCCCCCGAGCCCG
AGCCUGAGUGUUUCGGCAGCUUCAAGGUGAGCGACGUGGU
GGACGGCAACAUCCAGGGCCGGGUGAUGCUGAGCGGCAUG
GGCGAGGGAAAGAUCAGCGGGGGCGCCGCCGUGAGCGACA
GCAGCAGCGCCAGCAUGAACGUGUGCAUCCUGCGGGUGAC
CCAGAAGACCUGGGAGACCAUGCAGCACGAGCGGCACCUG
CAGCAGCCUGAGAAUAAGAUCCUGCAGCAGCUGCGGAGCC
GGGGCGAUGACCUGUUCGUGGUGACCGAAGUGCUGCAGAC
CAAGGAGGAGGUGCAGAUCACCGAGGUGCACAGCCAGGAG
GGCAGCGGCCAGUUCACCCUGCCCGGCGCCCUGUGCCUGA
AGGGCGAGGGCAAGGGACACCAGAGCCGGAAGAAGAUGGU
GACCAUCCCCGCCGGCAGCAUCCUGGCCUUCCGGGUGGCC
CAGCUGCUGAUCGGCAGCAAGUGGGACAUUCUGCUGGUGA
GCGACGAGAAGCAGCGGACCUUCGAGCCCAGCAGCGGCGA
CCGGAAGGCCGUGGGCCAGCGGCACCACGGCCUGAACGUG
CUGGCCGCCCUGUGUAGCAUCGGCAAGCAGCUAAGCCUGC
UGAGCGACUGAUUAAACCGGUCUGCAGCUUUUGCCUUGGU
UCUUCCUAGUGCCUACAAUGGGAAAACUUCACUCCAAAGA
GAAACCUAUUAAGUCAUCAUCUCCAAACUAAACCCUCACA
AGUAACAGUUGAAGAAAAAAUGGCAAGAGAUCAUAUCCUC
AGACCAGGUGGAAUUACUUAAAUUUUAAAGCCUGAAAAUU
CCAAUUUGGGGGUGGGAGGUGGAAAAGGAACCCAAUUUUC
UUAUGAACAGAUAUUUUUAACUUAAUGGCACAAAGUCUUA
GAAUAUUAUUAUGUGCCCCGUGUUCCCUGUUCUUCGUUGC
UGAAUUCGAUAUCCAGCGGCCGCGCUAGCGCUG

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of treating a subject, wherein the method comprises administering a therapeutically effective amount of an in-vitro transcribed mRNA encoding a constitutively active polypeptide.

2. The method of claim 1, wherein the subject is suffering from cancer.

3. The method of claim 1, wherein the polypeptide is a component of a cell death pathway.

4. The method of claim 1, wherein the polypeptide is a suicide protein.

5. The method of claim 1, wherein the polypeptide is MLKL.

6. The method of claim 1, wherein the polypeptide is gasdermin.

7. The method of claim 1, wherein the polypeptide is RIPK3.

8. The method of claim 1, wherein the polypeptide is constitutively active due to a gain of function mutation.

9. The method of claim 8, wherein the polypeptide is MLKL and the gain of function mutation is K230M/Q356A

10. The method of claim 1, wherein the polypeptide is the N-terminal domain of gasdermin.

11. The method of claim 1, wherein the in-vitro transcribed mRNA comprises an oncoselective readthrough element.

12. The method of claim 11, wherein the polypeptide is MLKL and the gain of function mutation is K230M/Q356A.

13. The method of claim 11, wherein the polypeptide is the N-terminal domain of gasdermin.

14. The method of claim 1, wherein the mRNA enters a cell in the subject and the constitutively active polypeptide is expressed.

15. The method of claim 14, wherein expression of the polypeptide induces cell death.

16. The method of claim 14, wherein expression of the polypeptide induces proinflammatory cell death.

17. The method of claim 14, wherein expression of the polypeptide induces immunogenic cell death.

18. The method of claim 14, wherein expression of the polypeptide induces pyroptosis.

19. The method of claim 14, wherein expression of the polypeptide induces necroptosis.

20. A pharmaceutical composition comprising an in-vitro transcribed mRNA encoding a constitutively active polypeptide.

21. The composition of claim 20, wherein the composition comprises nanoparticles.

22. The composition of claim 21, wherein the nanoparticles are lipid nanoparticles.

23. An in-vitro transcribed mRNA encoding a constitutively active polypeptide.

24. The mRNA of claim 23, wherein the polypeptide is a component of a cell death pathway.

25. The mRNA of claim 23, wherein the polypeptide is or comprises a suicide protein, or effective fragment or portion thereof.

26. The mRNA of claim 23, wherein the suicide protein is MLKL.

27. The mRNA of claim 23, wherein the suicide protein is gasdermin.

28. The mRNA of claim 23, wherein the polypeptide is RIPK3.

29. The mRNA of claim 23, wherein the polypeptide is constitutively active due to a gain of function mutation.

30. The mRNA of claim 29, wherein the suicide protein is MLKL and the gain of function mutation is K230M/Q356A.

31. The mRNA of claim 23, wherein the suicide protein is the N-terminal domain of gasdermin.
